(19) United States
(12) Patent Application Publication
(10) Pub. No.: US 2006/0025421 A1
(43) Pub. Date: Feb. 2, 2006

(54) Pyridyl piperazines for the treatment of CNS disorders

(75) Inventor: Michael A. Brodney, East Lyme, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/166,519

(22) Filed: Jun. 24, 2005

Related U.S. Application Data
(60) Provisional application No. 60/582,969, filed on Jun. 25, 2004.

(51) Int. Cl. A61K 31/496; C07D 413/14; C07D 403/14

(52) U.S. Cl. 514/253.09; 544/360

(57) ABSTRACT

This invention is directed to compounds of Formula I and to pharmaceutical compositions comprising the compound of Formula I. 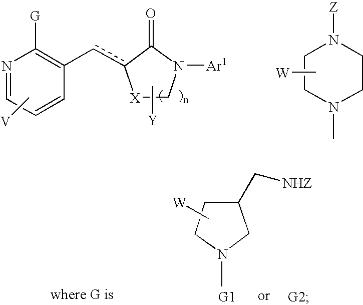
where the dashed line represents an optional double bond; and where n is 1 or 2, and Ar¹, Ar², . . . and Z are as defined in the specification. The invention is also directed to a method of treating a disorder or condition that can be treated by altering serotonin-mediated neurotransmission, such as migraine, headache, cluster headache, anxiety, depression, etc. This invention is also directed to intermediates useful in the synthesis of compounds of Formula I.

### FIELD OF THE INVENTION

This invention relates to pyridyl piperazines having affinity for serotonin (5HT) receptors, especially the serotonin IB receptor (5HT_{1B}), and to their use in treating diseases or conditions which are caused by disorders of the serotonin system.

### BACKGROUND OF THE INVENTION

Serotonin, also known as 5-hydroxytryptamine and abbreviated “5HT,” is ubiquitous in plants and animals and is implicated in a great many physiological pathways, both normal and pathological. It is an important neurotransmitter and local hormone both in the periphery, particularly the intestine, and in the central nervous system (CNS). In the periphery, 5HT contracts a number of smooth muscles, induces endothelium-dependent vasodilation through the formation of nitric oxide, mediates peristalsis, and may be involved in platelet aggregation and homeostasis. In the CNS, 5HT is believed to be involved in a wide range of functions, including the control of appetite, mood, anxiety, hallucinations, sleep, vomiting, and pain perception. (Watson, S. and Arkinstall, S. “5-Hydroxytryptamine” in The G Protein-Linked Receptor Factsbook, Academic Press, 1994, pp. 159-180.)

Serotonin plays a role in numerous psychiatric disorders, including anxiety, Alzheimer's disease, depression, nausea and vomiting, eating disorders, and migraine. (Rasmussen et al., “Chapter 1. Recent Progress in Serotonin (5HT), Receptor Modulators,” in Ann. Rep. Med. Chem., 1, 30, pp. 1-9, 1995, Academic Press). Serotonin also plays a role in both the positive and negative symptoms of schizophrenia. (Sharma et al., Psychiatric Ann., 1996, 26 (2), pp. 88-92.)

Several serotonin receptor subtypes have been classified according to their antagonist susceptibilities and their affinities for 5HT. The 5HT_{1B} receptor was first identified in rats, where it has a distinct pharmacological profile. In humans, however, it shares an almost identical pharmacology with the 5HT_{1D} receptor. In the CNS, the 5HT_{1B} receptor is found in the striatum, medulla, hippocampus, frontal cortex and amygdala. In the periphery, it is found in vascular smooth muscle. Therefore, in humans the receptor is often denoted the “5HT_{1B}/5HT_{1D} receptor.” The 5HT_{1B}/5HT_{1D} receptor may be the therapeutic substrate of the anti-migraine drug, sumatriptan; the 5HT_{1B}/5HT_{1D} receptor is also implicated in feeding behavior, anxiety, depression, cardiac function, and movement. (Watson, S. and Arkinstall, S. op. cit.)

The 5HT_{1B} receptor was the first subtype to have its gene inactivated by classical homologous recombination (Saudou F, et al., Science, 1994, 265, 1875-1878). 5HT_{1B} receptors are expressed in the basal ganglia, central gray, hippocampus, amygdala, and raphe nuclei. They are located predominantly at presynaptic terminals where they can inhibit release of 5HT and, as heteroceptors, of other neurotransmitters. Selective agonists and antagonists for 5HT_{1B} receptors have until now been lacking, but indirect pharmacological evidence suggests that 5HT_{1B} activation influences food intake, sexual activity, locomotion, and aggression. (Ramboz, S., et al., Behav. Brain Res. 1996 73: 305312.)

### SUMMARY OF THE INVENTION

This invention relates to certain pyridyl piperazines. These compounds are antagonists of the serotonin 5HT_{1B} receptor. As such, they are effective for the treatment of disorders of the serotonin system, such as depression and related disorders. In particular, the invention is directed to pyridyl piperazine compounds of Formula I: 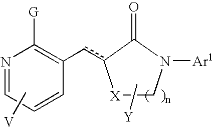
and to pharmaceutically acceptable salts and prodrugs thereof where G is 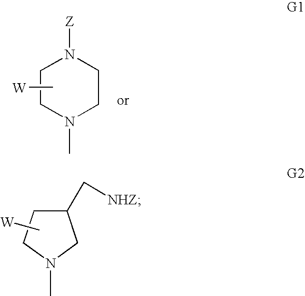
where the dashed line represents an optional double bond; where Ar¹ is phenyl, a 5- or 6-membered heteroaryl ring, or an 8- to 10-membered fused aryl or heteroaryl ring system, said heteroaryl ring, and the heteroaryl moiety of said heteroaryl ring system comprising an aromatic ring made up of carbon and from one to four other elements selected independently from the group consisting of oxygen, nitrogen, and sulfur, which Ar¹ may be singly or multiply substituted with, independently, halogen, hydroxy, nitro, cyano, R¹, R², R³, —OR⁴, —OC(═O)R⁵, —COOR⁶, NHR⁷, NR⁸R⁹, —NHC(═O)R¹⁰, N(R¹¹)(C═O)R¹², —C(═O)NHR¹³, or Ar²; X is CH₂, NH, or O; V, W, and Y are, independently, hydrogen, halogen, hydroxy, nitro, cyano or R⁷, where R¹-R¹³ are, independently, C₁-C₈ alkyl, C₂-C₈ alkenyl, C₁-C₈ alkoxy, C₁-C₈ hydroxyalkyl, C₁-C₈ alkenoxy, said alkyl, alkenyl, alkoxy, or alkenoxy optionally substituted with one or more halogen atoms or nitro, cyano, or hydroxyl groups, said alkyl or alkenyl groups being straight-chain, branched, or cyclic, wherein an alkoxy-substituted alkyl group may form a cyclic ether, or, in the case of NR⁸R⁹, R⁸ and R⁹, may be linked together to form an additional ring; Z is C₁-C₆ alkyl or C₁-C₆ alkylcarbonyl; Ar² is a 5- or 6-membered aryl or heteroaryl ring or an 8- to 10-membered fused aryl or heteroaryl ring system, which Ar² may be singly or multiply substituted with, independently, halogen, hydroxy, nitro, cyano, R¹, R², R³, OR⁴, OC(═O)R⁵, COOR⁶, NHR⁷, NR⁸R⁹, NHC(═O)R¹⁰, N(R¹¹)(C═O)R¹², C(═O)NHR¹³; and n is 1 or 2.

The invention is also directed to pharmaceutical compositions comprising the compound of Formula I, or a pharmaceutically acceptable salt or prodrug thereof, and a pharmaceutically effective carrier.

The invention is further directed to a method of treating or preventing a disorder or condition that can be treated by altering serotonin-mediated neurotransmission in a mammal, including a human.

The invention is still further directed to a method of treating, in a mammal, including a human, a disorder selected from the group consisting of anxiety, depression, dysthymia, major depressive disorder, migraine, post-traumatic stress disorder, avoidant personality disorder, borderline personality disorder, and phobias comprising administering to a mammal or human in need thereof a treatment effective amount of the compound of Formula I, or a pharmaceutically acceptable salt or prodrug thereof.

The invention is also directed to any of the foregoing methods wherein the compound of Formula I, or a pharmaceutically acceptable salt thereof, is administered in combination with a serotonin reuptake inhibitor (SRI) (e.g., sertraline, fluoxetine, fenfluramine, or fluvoxamine). The term “administered in combination with,” as used herein, means that the compound of Formula I or pharmaceutically acceptable salt thereof is administered in the form of a pharmaceutical composition that also contains an SRI, or that such compound or salt is administered in a separate pharmaceutical composition from that in which the SRI is administered, but as part of a dosage regimen that calls for the administration of both active agents for treatment of a particular disorder or condition.

The terms “pharmaceutically acceptable salts” and “pharmaceutically acceptable acid salts” of compounds of the Formula I refer to those salts which are, within the scope of sound medical judgment, suitable for use in contact with the tissues of patients without undue toxicity, irritation, allergic response, and the like, as well as zwitterionic forms, where possible of compounds of the invention. The compounds of Formula I are basic in nature and are thus capable of forming a wide variety of salts with various inorganic and organic acids. The acids that can be used to prepare pharmaceutically acceptable acid addition salts of those compounds of Formula I are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, fumarate, gluconate, glucaronate, saccharate, formate, benzoate, glutamate, methanesulfonate, ethanesulfonate, benzenesulfonate, and p-toluenesulfonate. (See, for example, Berge, S. M., et al., “Pharmaceutical Salts,” J. Pharm. Sci. (1977) vol. 66, pp. 1-19, which is incorporated herein by reference.)

The term “one or more substituents,” as used herein, includes from one to the maximum number of substituents possible based on the number of available bonding sites.

The term “disorders of the serotonin system,” as used herein, refers to disorders the treatment of which can be effected or facilitated by altering (i.e., increasing or decreasing) serotonin-mediated neurotransmission.

The term “treating,” as used herein, refers to retarding or reversing the progress of, or alleviating or preventing either the disorder or condition to which the term “treating” applies, or one or more symptoms of such disorder or condition. The term “treatment,” as used herein, refers to the act of treating a disorder or condition, as the term “treating” is defined above.

The term “treatment effective amount,” as used herein, refers to an amount sufficient to detectably treat, ameliorate, prevent or detectably retard the progression of an unwanted condition or symptom associated with disorders of the serotonin system.

The term “serotonin-mediated neurotransmission-altering effective amount,” as used herein, refers to an amount sufficient to increase or decrease neurotransmission in systems controlled by serotonin.

The term “prodrug,” as used herein, refers to a chemical compound that is converted by metabolic processes in vivo to a compound of the above formula. An example of such a metabolic process is hydrolysis in blood. Thorough discussions of prodrugs are provided in T. Higuchi and V. Stella, “Prodrugs as Novel Delivery Systems,” Vol. 14, ACS Symposium Series, and in “Bioreversible Carriers in Drug Design,” ed. Edward Roche, American Pharmaceutical Association and Pergamon Press, 1987, both of which are incorporated herein by reference.

The chemist of ordinary skill will recognize that certain combinations of substituents, included within the scope of formula I, may be chemically unstable. The skilled chemist will either avoid these combinations or protect sensitive groups with well-known protecting groups.

The term “alkyl,” as used herein, unless otherwise indicated, includes saturated monovalent hydrocarbon radicals with 1-12 carbon atoms having straight, branched or cyclic moieties or combinations thereof. The term “lower alkyl” refers to an alkyl group having one to six carbon atoms. Examples include methyl, ethyl, propyl, isopropyl, cyclopropyl, butyl, isobutyl, sec-butyl, t-butyl, pentyl, neopentyl, cyclopentylmethyl, and hexyl. It is preferred that the alkyl group is lower alkyl. The preferred cyclic alkyl groups are cyclobutyl and cyclopentyl. The preferred lower alkyl group contains 1-3 carbon atoms. The most preferred alkyl group is methyl.

The term “alkoxy,” as used herein, unless otherwise indicated, refers to radicals having the formula —O-alkyl, wherein “alkyl” is defined as above. As used herein, the term “lower alkoxy” refers to an alkoxy group having 1-6 carbon atoms. It may be straight-chain or branched or an alkoxy-substituted alkyl group may form a cyclic ether, such as tetrahydropyran or tetrahydrofuran. Examples of acyclic alkoxy groups include methoxy, ethoxy, propoxy, isopropoxy, butoxy, isobutoxy, pentoxy and the like. It is preferred that alkoxy is lower alkoxy. It is more preferred that alkoxy contains 1:3 carbon atoms. The most preferred alkoxy group is methoxy. The most preferred substituted alkoxy group is trifluoromethoxy.

The halogen atoms contemplated by the present invention are F, Cl, Br, and I. Chlorine and fluorine are preferred. Alkyl groups substituted with one or more halogen atoms include chloromethyl, 2,3-dichloropropyl, and trifluoromethyl. It is preferred that the halo groups are the same. The most preferred halogen-substituted alkyl group is trifluoromethyl.

The term “alkenyl,” as used herein, refers to a hydrocarbon radical with two to eight carbon atoms and at least one double bond. The alkenyl group may be straight-chained, branched, or cyclic, and may be in either the Z or E form. Examples include ethenyl, 1-propenyl, 2-propenyl, 1-butenyl, 2-butenyl, isopropenyl, isobutenyl, 1-pentenyl, (Z)-2-pentenyl, (E)-2-pentenyl, (Z)-4-methyl-2-pentenyl, (E)-4-methyl-2-pentenyl, 1,3-pentadienyl, 2,4-pentadienyl, 1,3-butadienyl, cyclopentadienyl, and the like. The preferred alkenyl group is ethenyl.

The term “alkynyl, as used herein,” refers to a hydrocarbon radical with two to eight carbon atoms and at least one carbon-carbon triple bond. The alkynyl group may be straight chained or branched. Examples include 1-propynyl, 2-propynyl, 1-butynyl, 2-butynyl, 1-pentynyl, 2-pentynyl, 3-methyl-1-pentynyl, 3-pentynyl, 1-hexynyl, 2-hexynyl, 3-hexynyl, and the like. The preferred alkynyl group is ethynyl.

The term “aryl,” as used herein, unless otherwise indicated, includes an organic radical derived from a C₆-C₁₄ aromatic hydrocarbon by removal of one or more hydrogen(s). Examples include phenyl and naphthyl. The preferred substitution pattern of the phenyl group is para.

The term “heteroaryl,” as used herein, unless otherwise indicated, includes an organic radical derived from an aromatic heterocyclic compound by removal of one or more hydrogen atoms. The term “heterocyclic compound” denotes a ring system made up of 5-14 ring atoms and made up of carbon and at least one other element selected from the group consisting of oxygen, nitrogen, and sulfur. Examples of heteroaryl groups include benzimidazolyl, benzofuranyl, benzofurazanyl, 2H-1-benzopyranyl, benzothiadiazine, benzothiazinyl, benzothiazolyl, benzothiophenyl, benzoxazolyl, furazanyl, furopyridinyl, furyl, imidazolyl, indazolyl, indolinyl, indolizinyl, indolyl, 3H-indolyl, isoindolyl, isoquinolinyl, isothiazolyl, isoxazolyl, naphthyridinyl, oxadiazolyl, oxazolyl, phthalazinyl, pteridinyl, purinyl, pyrazinyl, pyridazinyl, pyridinyl, pyrimidinyl, pyrazolyl, pyrrolyl, quinazolinyl, quinolinyl, quinoxalinyl, tetrazolyl, thiazolyl, thiadiazolyl, thienyl, triazinyl and triazolyl. Preferred heteroaryl groups are oxazolyl and isoxazolyl.

The compounds of Formula I contain one or more chiral centers and therefore exist in different enantiomeric and diasteriomeric forms. Formula I, as defined above, includes—and this invention relates to the use of—all optical isomers and other stereoisomers of compounds of Formula I and mixtures thereof. Where compounds of this invention exist in different tautomeric forms, this invention relates to all tautomers of Formula I.

Preferred compounds of this invention are those wherein V, W, and Y are hydrogen, Z is methyl, and the dashed line in Formula I is a single bond.

Other preferred compounds of this invention are those in which X is CH₂ or O. Most preferred are those in which X is CH₂.

Other preferred compounds of this invention are those in which G is 4-methyl-piperazin-1-yl.

Specific preferred compounds of formula I are:
1-[4-(3,5-Dimethyl-isoxazol-4-yl)phenyl]-3-[2-(4-methyl-piperazin-1-yl)-pyridin-3-ylmethylene]-piperidin-2-one;
2-[2-(4-Methyl-piperazin-1-yl)-pyridin-3-ylmethylene]-4-[4-(tetrahydro-pyran-4-yl)-phenyl]-morpholin-3-one;
1-[4-(3,5-Dimethyl-isoxazol-4-yl)phenyl]-3-[2-(4-methyl-piperazin-1-yl)-pyridin-3-ylmethyl]-piperidin-2-one;
2-[2-(4-Methyl-piperazin-1-yl)-pyridin-3-ylmethyl]-4-[4-(tetrahydro-pyran-4-yl)-phenyl]-morpholin-3-one;
1-[4-(2-Methyl-oxazol-4-yl)-phenyl]-3-[2-(4-methyl-piperazin-1-yl)-pyridin-3-ylmethyl]-piperidin-2-one;
1-[4-(2-tert-Butyl-oxazol-4-yl)phenyl]-3-[2-(4-methyl-piperazin-1-yl)-pyridin-3-ylmethyl]-piperidin-2-one;
1-[4-(2-Isopropyl-oxazol-4-yl)phenyl]-3-[2-(4-methyl-piperazin-1-yl)pyridin-3-ylmethyl]-piperidin-2-one;
1-[4-(2,5-Dimethyl-oxazol-4-yl)-phenyl]-3-[2-(4-methyl-piperazin-1-yl)-pyridin-3-ylmethyl]-piperidin-2-one;
3-[2-(4-Methyl-piperazin-1-yl)-pyridin-3-ylmethyl]-1-[4-(tetrahydro-pyran-4-yl)-phenyl]-piperidin-2-one;
3-[2-(4-Methyl-piperazin-1-yl)-pyridin-3-ylmethyl]-1-[4-(tetrahydro-pyran-4-yl)-phenyl]-pyrrolidin-2-one;
3-[2-(4-Methyl-piperazin-1-yl)-pyridin-3-ylmethyl]-1-(4-oxazol-2-yl-phenyl)-pyrrolidin-2-one;
3-[2-(4-Methyl-piperazin-1-yl)-pyridin-3-ylmethyl]-1-(4-oxazol-4-yl-phenyl)pyrrolidin-2-one;
3-[2-(4-Methyl-piperazin-1-yl)-pyridin-3-ylmethyl]-1-(4-oxazol-5-yl-phenyl)-pyrrolidin-2-one;
1-[4-(2-Methyl-oxazol-4-yl)phenyl]-3-[2-(4-methyl-piperazin-1-yl)-pyridin-3-ylmethyl]-pyrrolidin-2-one;
1-[4-(1-Methoxy-cyclobutyl)-phenyl]-3-[2-(4-methyl-piperazin-1-yl)-pyridin-3-ylmethyl]-piperidin-2-one;
3-[2-(4-Methyl-piperazin-1-yl)-pyridin-3-ylmethyl]-1-(4-trifluoromethyl-phenyl)-pyrrolidin-2-one;
1-[4-(1-Hydroxy-cyclopentyl)phenyl]-3-[2-(4-methyl-piperazin-1-yl)-pyridin-3-ylmethyl]-pyrrolidin-2-one;
1-[4-(1-Hydroxy-1-methyl-ethyl)-phenyl]-3-[2-(4-methyl-piperazin-1-yl)pyridin-3-ylmethyl]-pyrrolidin-2-one;
1-(4-tert-Butyl-phenyl)-3-[2-(4-methyl-piperazin-1-yl)-pyridin-3-ylmethyl]-pyrrolidin-2-one;
1-[4-(1-Hydroxy-cyclopentyl)-phenyl]-3-[2-(4-methyl-piperazin-1-yl)-pyridin-3-ylmethyl]-piperidin-2-one;
1-(4-tert-Butyl-phenyl)-3-[2-(4-methyl-piperazin-1-yl)-pyridin-3-ylmethyl]-piperidin-2-one;
3-[2-(4-Methyl-piperazin-1-yl)-pyridin-3-ylmethyl]-1-phenyl-piperidin-2-one;
3-[2-(4-Methyl-piperazin-1-yl)-pyridin-3-ylmethyl]-1-(4-trifluoromethoxy-phenyl)-piperidin-2-one;
1-[4-(1-Hydroxy-1-methyl-ethyl)phenyl]-3-[2-(4-methyl-piperazin-1-yl)-pyridin-3-ylmethyl]-piperidin-2-one;
1-[4-1-Hydroxy-cyclobutyl)-phenyl]-3-[2-(4-methyl-piperazin-1-yl)-pyridin-3-ylmethyl]-piperidin-2-one;
1-(4-tert-Butyl-phenyl)-3-[2-(4-Methyl-piperazin-1-yl)-pyridin-3-ylmethyl]-pyrrolidin-2-one;
1-(4-tert-Butyl-phenyl)-3-[2-(4-Methyl-piperazin-1-yl)-pyridin-3-ylmethyl]-piperidin-2-one;
3-[2-(4-Methyl-piperazin-1-yl)-pyridin-3-ylmethyl]-1-(4-tetrahydropyran-4-yl)-phenyl]-piperidin-2-one;
1-[4-(1-Hydroxy-1-methyl-ethyl)-phenyl]-3-[2-(4-methyl-piperazin-1-yl)pyridin-3-ylmethyl]-piperidin-2-one; and
1-[4-(1-Hydroxy-1-methyl-ethyl)phenyl]-3-[2-(4-methyl-piperazin-1-yl)-pyridin-3-ylmethyl]-pyrrolidin-2-one.

This invention is also directed to an intermediate useful in the synthesis of a compound of Formula I, where the intermediate is selected from 3-[2-(4-Methyl-piperazin-1-yl)pyridin-3-ylmethylene]-pyrrolidin-2-one and 3-[2-(4-Methyl-piperazin-1-yl)-pyridin-3-ylmethylene]-piperidin-2-one.

5HT receptor ligands of the present invention are of clinical use in the treatment of a wide variety of disorders related to serotonin-mediated physiological pathways. Accordingly, this invention is directed to a method of treating a disorder or condition that can be treated by altering (i.e., increasing or decreasing) serotonin-mediated neuro-transmission in a mammal, including a human, comprising administering to said mammal an amount of a compound of the Formula I, as defined above, or a pharmaceutically acceptable salt thereof, that is effective in treating such disorder or condition.

This invention is also directed to a method of treating migraine, headache or cluster headache in a mammal, including a human, comprising administering to said mammal an amount of a compound of the Formula I, as defined above, or a pharmaceutically acceptable salt thereof, that is effective in treating such disorder.

This invention is also directed to a method of treating a disorder selected from, depression (i.e., dysthymia, major depressive disorder, pediatric depression, recurrent depression, single episode depression, post partum depression, depression in Parkinson's patients, cancer patients, and post myocardial infarction patients, and subsyndromal symptomatic depression) generalized anxiety disorder, panic disorder, obsessive-compulsive disorder, post-traumatic stress disorder, avoidant personality disorder, borderline personality disorder and phobias in a mammal, including a human, comprising administering to said mammal an amount of a compound of the formula I, as defined above, or a pharmaceutically acceptable salt thereof, that is effective in treating such disorder.

Formula I above includes compounds identical to those depicted but for the fact that one or more atoms (for example, hydrogen, carbon, or fluorine atoms) are replaced by radioactive isotopes thereof. Such radiolabelled compounds are useful as research and diagnostic tools in, for example, metabolism studies, pharmacokinetic studies and binding assays.

This invention is also directed to a method, such as positron emission tomography (PET), of obtaining images of a mammal, including a human, to which a radiolabelled compound of the Formula I, or pharmaceutically acceptable salt thereof, has been administered. Such imaging methods can be used for any organ or system in which the 5-HT_{1B} receptor is found, such as those indicated above. The utility of radioactive agents with affinity for 5HT receptors for visualizing organs of the body either directly or indirectly has been documented in the literature. For example, C.-Y. Shiue et al., Synapse, 1997, 25, 147 and S. Houle et al., Can. Nucl. Med. Commun., 1997, 18, 1130, describe the use of 5HT_{1A} receptor ligands to image 5HT_{1A} receptors in the human brain using positron emission tomography (PET). The foregoing references are incorporated herein by reference in their entireties.

The compounds of Formula I and their pharmaceutically acceptable salts can be prepared as described below.

Compounds of Formula I in which one or more atoms are radioactive can be prepared by methods known to a person of ordinary skill in the art. For example, compounds of Formula I wherein the radioactive atom is tritium can be prepared by reacting an aryl halide Ar—X, wherein the halogen is chlorine, bromine or iodine, with gaseous ³H₂ and a noble metal catalyst, such as palladium suspended on carbon, in a suitable solvent such as a lower alcohol, preferably methanol or ethanol. Compounds of Formula I wherein the radioactive atom is ¹⁸F can be prepared by reacting an aryl trialkyl stannane Ar—SnR³, wherein R is lower alkyl, preferably methyl or n-butyl, with ¹⁸F-enriched fluorine (F₂), OF₂ or CF₃COOH in a suitably inert solvent (c.f M. Namavari, et al., J. Fluorine Chem., 1995, 74, 113).

Compounds of Formula I wherein the radioactive atom is ¹⁴C can be prepared by reacting an aryl halide Ar—X, wherein X is preferably bromine or iodine, or an aryl trifluoromethane sulfonate (Ar—OSO₂CF₃) with potassium [¹⁴C]cyanide or potassium [¹⁴C]-cyanide and a noble metal catalyst, preferably tetrakis(triphenylphosphine)palladium, in a reaction inert solvent such water or tetrahydrofuran, and preferably a mixture of water and tetrahydrofuran. (See Y. Andersson, B. Langstrom, J. Chem. Soc. Perkin Trans. 1, 1994, 1395.)

The therapeutic compounds used in the methods of this invention can be administered orally, buccally, transdermally (e.g., through the use of a patch), parenterally or topically. Oral administration is preferred. In general, these compounds are most desirably administered in dosages ranging from about 1 mg to about 1000 mg per day, although variations may occur depending on the weight and condition of the person being treated and the particular route of administration chosen. In some instances, dosage levels below the lower limit of the aforesaid range may be more than adequate, while in other cases still larger doses can be employed without causing any harmful side effects, provided that such larger doses are first divided into several small doses for administration throughout.

The compounds of this invention can be used in combination with a serotonin re-uptake inhibitor (SRI). When used in the same oral, parenteral or buccal pharmaceutical composition as an SRI, the daily dose of the compound of formula I or pharmaceutically acceptable salt thereof will be within the same general range as specified above for the administration of such compound or salt as a single active agent. The daily dose of the SRI in such a composition will generally be within the range of about 1 mg to about 400 mg

The therapeutic compounds used in the methods of this invention can be administered alone or in combination with pharmaceutically acceptable carriers or diluents by either of the two routes previously indicated, and such administration can be carried out in single or multiple doses. More particularly, the therapeutic compounds used in the methods of this invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Such carriers include solid diluents or fillers, sterile aqueous media and various non-toxic organic solvents, for example. Moreover, oral pharmaceutical compositions can be suitably sweetened and/or flavored.

For oral administration, tablets containing various excipients such as microcrystalline cellulose, sodium citrate, calcium carbonate, dicalcium phosphate and glycine can be employed along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tabletting purposes. Solid compositions of a similar type can also be employed as fillers in gelatin capsules; preferred materials in this connection also include lactose or milk sugar as well as high molecular weight polyethylene glycols. When aqueous suspensions and/or elixirs are desired for oral administration, the active ingredient can be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a therapeutic compound used in the methods of the present invention in either sesame or peanut oil or in aqueous propylene glycol can be employed. The aqueous solutions should be suitably buffered if necessary and the liquid diluent first rendered isotonic. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

### Biological Assay

The activity of the compounds of the present invention with respect to 5HT_{1B} (formerly referred to as 5HT_{1D}) binding ability can be determined using standard radioligand binding assays as described in the literature. The 5-HT_{1A} affinity can be measured using the procedure of Hoyer et al. (Brain Res., 1986, 376, 85). The 5-HT_{1D} affinity can be measured using the procedure of Heuring and Peroutka (J. Neurosci., 1987, 7, 894).

The in vitro activity of the compounds of the present invention at the 5-HT_{1D} binding site may be determined according to the following procedure. Bovine caudate tissue is homogenized and suspended in 20 volumes of a buffer containing 50 mM TRIS.hydrochloride (tris[hydroxymethyl]aminomethane hydrochloride) at a pH of 7.7. The homogenate is then centrifuged at 45,000 G for 10 minutes. The supernatant is then discarded and the resulting pellet resuspended in approximately 20 volumes of 50 mM TRIS-hydrochloride buffer at pH 7.7. This suspension is then pre-incubated for 15 minutes at 37° C., after which the suspension is centrifuged again at 45,000 G for 10 minutes and the supernatant discarded. The resulting pellet (approximately 1 gram) is resuspended in 150 ml of a buffer of 15 mM TRIS-hydrochloride containing 0.01 percent ascorbic acid with a final pH of 7.7 and also containing 10 μM pargyline and 4 mM calcium chloride (CaCl₂). The suspension is kept on ice at least 30 minutes prior to use.

The inhibitor, control or vehicle is then incubated according to the following procedure. To 50 μl of a 20 percent dimethylsulfoxide (DMSO)/80 percent distilled water solution is added 200 μl of tritiated 5-hydroxytryptamine (2 nM) in a buffer of 50 mM TRIS.hydrochloride containing 0.01 percent ascorbic acid at pH 7.7 and also containing 10 μM pargyline and 4 μM calcium chloride, plus 100 nM of 8-hydroxy-DPAT (dipropylaminotetraline) and 100 nM of mesulergine. To this mixture is added 750 μl of bovine caudate tissue, and the resulting suspension is vortexed to ensure a homogenous suspension. The suspension is then incubated in a shaking water bath for 30 minutes at 25° C. After incubation is complete, the suspension is filtered using glass fiber filters (e.g., Whatman GF/B-filters.™.). The pellet is then washed three times with 4 ml of a buffer of 50 mM TRIS.hydrochloride at pH 7.7. The pellet is then placed in a scintillation vial with 5 ml of scintillation fluid (aquasol 2™) and allowed to sit overnight. The percent inhibition can be calculated for each dose of the compound. An IC₅₀ value can then be calculated from the percent inhibition values.

The activity of the compounds of the present invention for 5-HT_{1A} binding ability can be determined according to the following procedure. Rat brain cortex tissue is homogenized and divided into samples of 1 gram lots and diluted with 10 volumes of 0.32 M sucrose solution. The suspension is then centrifuged at 900 G for 10 minutes and the supernate separated and recentrifuged at 70,000 G for 15 minutes. The supernate is discarded and the pellet re-suspended in 10 volumes of 15 mM TRIS.hydrochloride at pH 7.5. The suspension is allowed to incubate for 15 minutes at 37° C. After pre-incubation is complete, the suspension is centrifuged at 70,000 G for 15 minutes and the supernate discarded. The resulting tissue pellet is resuspended in a buffer of 50 mM TRIS.hydrochloride at pH 7.7 containing 4 mM of calcium chloride and 0.01 percent ascorbic acid. The tissue is stored at −70° C. until ready for an experiment. The tissue can be thawed immediately prior to use, diluted with 10 μm pargyline and kept on ice.

The tissue is then incubated according to the following procedure. Fifty microliters of control, inhibitor, or vehicle (1 percent DMSO final concentration) is prepared at various dosages. To this solution is added 200 μl of tritiated DPAT at a concentration of 1.5 nM in a buffer of 50 mM TRIS.hydrochloride at pH 7.7 containing 4 mM calcium chloride, 0.01 percent ascorbic acid and pargyline. To this solution is then added 750 μl of tissue and the resulting suspension is vortexed to ensure homogeneity. The suspension is then incubated in a shaking water bath for 30 minutes at 37° C. The solution is then filtered, washed twice with 4 ml of 10 mM TRIS.hydrochloride at pH 7.5 containing 154 mM of sodium chloride. The percent inhibition is calculated for each dose of the compound, control or vehicle. IC₅₀ values are calculated from the percent inhibition values.

The agonist and antagonist activities of the compounds of the invention at 5-HT_{1A} and 5-HT_{1D} receptors can be determined using a single saturating concentration according to the following procedure. Male Hartley guinea pigs are decapitated and 5-HT_{1A} receptors are dissected out of the hippocampus, while 5-HT receptors are obtained by slicing at 350 mM on a McIlwain tissue chopper and dissecting out the substantia nigra from the appropriate slices. The individual tissues are homogenized in 5 mM HEPES buffer containing 1 mM EGTA (pH 7.5) using a hand-held glass-Teflon® homogenizer and centrifuged at 35,000×g for 10 minutes at 4° C. The pellets are resuspended in 100 mM HEPES buffer containing 1 mM EGTA (pH 7.5) to a final protein concentration of 20 mg (hippocampus) or 5 mg (substantia nigra) of protein per tube. The following agents are added so that the reaction mix in each tube contained 2.0 mM MgCl₂, 0.5 mM ATP, 1.0 mM cAMP, 0.5 mM IBMX, 10 mM phosphocreatine, 0.31 mg/mL creatine phosphokinase, 100 μM GTP and 0.5-1 microcuries of [³²P]-ATP (30 Ci/mmol: NEG-003—New England Nuclear). Incubation is initiated by the addition of tissue to siliconized microfuge tubes (in triplicate) at 30° C. for 15 minutes. Each tube receives 20 μL tissue, 10 μL drug or buffer (at 10× final concentration), 10 μL 32 nM agonist or buffer (at 10× final concentration), 20 μL forskolin (3 μM final concentration) and 40 μL of the preceding reaction mix. Incubation is terminated by the addition of 100 μL 2% SDS, 1.3 mM cAMP, 45 mM ATP solution containing 40,000 dpm [³H]-cAMP (30 Ci/mmol: NET-275—New England Nuclear) to monitor the recovery of CAMP from the columns. The separation of [³²P]-ATP and [³²P]-cAMP is accomplished using the method of Salomon et al., Analytical Biochemistry, 1974, 58, 541-548. Radioactivity is quantified by liquid scintillation counting. Maximal inhibition is defined by 10 μM (R)-8-OH-DPAT for 5-HT_{1A} receptors, and 320 nM 5-HT for 5-HT_{1D} receptors. Percent inhibitions by the test compounds are then calculated in relation to the inhibitory effect of (R)-8-OH-DPAT for 5-HT_{1A} receptors or 5-HT for 5-HT_{1D} receptors. The reversal of agonist induced inhibition of forskolin-stimulated adenylate cyclase activity is calculated in relation to the 32 nM agonist effect.

The compounds of the invention can be tested in vivo for antagonism of 5-HT_{1D} agonist-induced hypothermia in guinea pigs according to the following procedure.

Male Hartley guinea pigs from Charles River, weighing 250-275 grams on arrival and 300-600 grams at testing, serve as subjects in the experiment. The guinea pigs are housed under standard laboratory conditions on a 7 a.m. to 7 p.m. lighting schedule for at least seven days prior to experimentation. Food and water are available ad libitum until the time of testing.

The compounds of the invention can be administered as solutions in a volume of 1 ml/kg. The vehicle used is varied depending on compound solubility. Test compounds are typically administered either sixty minutes orally (p.o.) or 0 minutes subcutaneously (s.c.) prior to a 5-HT_{1D} agonist, such as [3-(1-methylpyrrolidin-2-ylmethyl)-1H-indol-5-yl]-(3-nitropyridin-3-yl)-amine, which can be prepared as described in PCT Publication WO93/11106, published Jun. 10, 1993, the contents of which are incorporated herein by reference in its entirety, and which is administered at a dose of 5.6 mg/kg, s.c. Before a first temperature reading is taken, each guinea pig is placed in a clear plastic shoe box containing wood chips and a metal grid floor and allowed to acclimate to the surroundings for 30 minutes. Animals are then returned to the same shoe box after each temperature reading. Prior to each temperature measurement, each animal is firmly held with one hand for a 30-second period. A digital thermometer with a small animal probe is used for temperature measurements. The probe is made of semi-flexible nylon with an epoxy tip. The temperature probe is inserted 6 cm. into the rectum and held there for 30 seconds or until a stable recording is obtained. Temperatures are then recorded.

In p.o. screening experiments, a “pre-drug” baseline temperature reading is made at −90 minutes, the test compound is given at −60 minutes and an additional −30 minute reading is taken. The 5-HT_{1D} agonist is then administered at 0 minutes and temperatures are taken 30, 60, 120 and 240 minutes later. In subcutaneous screening experiments, a pre-drug baseline temperature reading is made at −30 minutes. The test compound and 5-HT_{1D} agonists are given concurrently and temperatures are taken at 30, 60, 120 and 240 minutes later.

Data are analyzed with two-way analysis of variants with repeated measures in Newman-Keuls post hoc analysis.

The active compounds of the invention can be evaluated as anti-migraine agents by testing the extent to which they mimic sumatriptan in contracting the dog isolated saphenous vein strip (P. P. A. Humphrey et al., Br. J. Pharmacol., 1988, 94, 1128). This effect can be blocked by methiothepin, a known serotonin antagonist. Sumatriptan is known to be useful in the treatment of migraine and produces a selective increase in carotid vascular resistance in the anesthetized dog. The pharmacological basis of sumatriptan efficacy has been discussed in W. Fenwick et al., Br. J. Pharmacol., 1989, 96, 83.

The serotonin 5-HT₁ agonist activity can be determined by the in vitro receptor binding assays, as described for the 5-HT_{1A} receptor using rat cortex as the receptor source and [³H]-8-OH-DPAT as the radioligand (D. Hoyer et al., Eur. J. Pharm., 1985, 118, 13) and as described for the 5-HT_{1D} receptor using bovine caudate as the receptor source and [³H]serotonin as the radioligand (R. E. Heuring and S. J. Peroutka, J. Neuroscience, 1987, 7, 894).

### DETAILED DESCRIPTION OF THE INVENTION

Scheme 1 illustrates general methods suitable for preparing compounds of formula I wherein X is carbon.

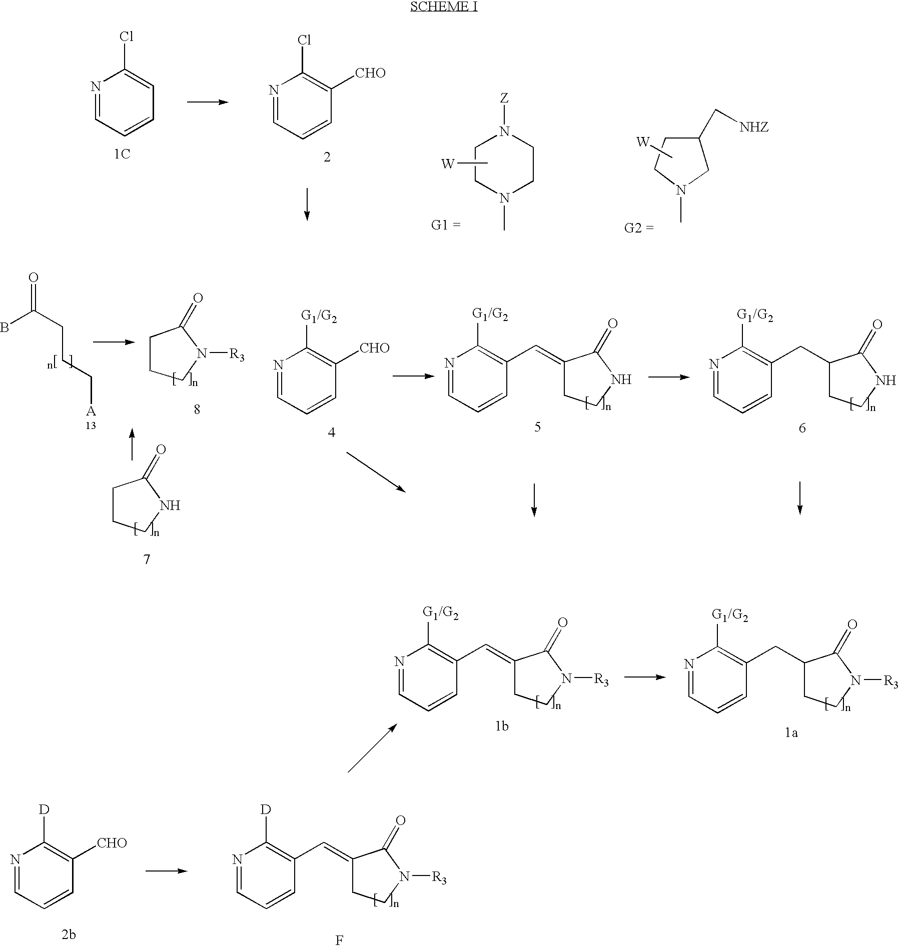

Synthesis of aldehyde 2 from 1C involves treatment of 1C with a tertiary amine, preferably N,N′-tetramethyl ethlyenediamine or 1,4-diazabicyclo[2.2.2]-octane, with a lithium alkyl base, preferably butyl lithium, in an ether solvent, preferably diethyl ether, at a temperature from about −100° to −30° C., preferably −78° C. Quenching with dimethylformamide at reaction temperature from about −100° to −30° C., where −78° C. is preferred, affords aldehyde 2.

Pyridyl piperazinyl aldehyde 4 is produced by the reaction of compound 2,2-chloro-pyridine-3-carbaldehyde, with G1* or G2* in the presence of a base such as a trialkyl amine or an alkali metal carbonate (a base that is inert towards 2, G1 or, and the solvent) in a solvent such as water, 1,4-dioxane, n-butanol, N,N-dimethyl-formamide, or dimethyl sulfoxide, at reaction temperature from about 40° to 150° C. The preferred base is potassium carbonate, the preferred solvent is water, and the preferred temperature is from about 90° to about 120° C.

Condensation of 4 and N-substituted lactam 8, in the presence of an amine or metal hydride base affords 5. The N-substituent (R3) can be vinyl or C(═O)R, (wherein R=C₁-C₈ alkyl-straight chain, branched or (if C₃-C₈) cyclic, or aryl). R=tert-butyl is preferred (Sasaki, H. et al. J. Med. Chem., 1991, 34, 628-633). The base can be sodium hydride or sodium bis(trimethylsilylamide), where sodium bis(trimethylsilylamide) is preferred. The preferred solvent is tetrahydrofuran. The reaction temperature is from about −30° to 100° C., preferably from about −10° to about 30° C. Reduction of the carbon-carbon double bond of 5 to generate 6 can be achieved by placing 5 in a reaction inert solvent such as a lower alcohol, wherein methanol or ethanol are preferred, adding a noble metal catalyst suspended on a solid support, such as platinum or palladium, where 10% palladium on carbon is preferred, then placing the mixture under a hydrogen atmosphere, from about 1 atm to 5 atm, where about 3 to about 4 atm is preferred, at a temperature from about 10° to 100° C., where 40° to 60° C. is preferred, and then shaking the mixture. In the case where R⁶=benzyl or some other group that is labile under hydrogenation conditions, the corresponding NH derivative (R⁶=H) is formed.

The conversion of 6 to 1a, wherein R³ is an optionally substituted aryl or heteroaryl group, can be accomplished by treating 6, an aryl or heteroaryl chloride, bromide, iodide, or sulfonate, where the bromide is preferred, a base such as potassium phosphate, potassium carbonate, sodium carbonate, thallium carbonate, cesium carbonate, potassium tert-butoxide, lithium tert-butoxide, or sodium tert-butoxide, where potassium carbonate is preferred, a diamine, such as 1,2-ethylenediamine, N,N′-dimethyl ethylenediamine, or cis-1,2-diaminocyclohexane, where N,N′-dimethylethylene-diamine is preferred, a cuprous chloride, bromide or iodide, where cuprous iodide is preferred, a small amount of water, where about 1 to 4 percent is preferred, in a reaction inert solvent such as 1,2-dimethoxyethane, diglyme, t-butyl methyl ether, tetrahydrofuran, benzene, toluene, where toluene is preferred, from about 40° to about 150° C., where about 80° to 120° C. is preferred. Alternatively, the conversion of 6 to 1a, wherein R³ is an optionally substituted aryl or heteroaryl group, can be accomplished by treating 6 and an aryl or heteroaryl chloride, bromide, iodide, or sulfonate, where the bromide is preferred, with a base such as an alkali metal carbonate, an alkali metal amine base, an alkali metal phosphonate, or an alkali metal alkoxide, where cesium carbonate is preferred, a phosphine ligand, where 9,9-dimethyl-4,5-bis(diphenyl-phosphino)xanthene (XANTPHOS) is preferred, a palladium species, such as palladium (II) acetate or tris(dibenzylidene-acetone)dipalladium (0) or the corresponding chloroform adduct, where tris(dibenzylidene-acetone)dipalladium (0) is preferred, in an inert solvent such as 1,4-dioxane or toluene, where 1,4-dioxane is preferred, at a temperature from about 40° to about 160° C., where 80° to 120° C. is preferred. If R⁶=H, then further functionalization of the secondary amine can be carried out under standard alkylation or reductive amination conditions known to one skilled in the art.

Another route to access compounds of formula 1a and 1b is shown in Scheme 1. The conversion of 5 to 1b, wherein R³ is Ar¹, an optionally substituted aryl or heteroaryl group as described above, can be accomplished by treating 5, an aryl or heteroaryl chloride, bromide, iodide, or sulfonate, where the bromide is preferred, a base such as potassium phosphate, potassium carbonate, sodium carbonate, thallium carbonate, cesium carbonate, potassium tert-butoxide, lithium tertbutoxide, or sodium tertbutoxide, where potassium carbonate is preferred, a diamine, such as 1,2-ethylenediamine, N,N′-dimethyl-ethylenediamine, or cis-1,2-diaminocyclohexane, where N,N′-dimethylethylenediamine is preferred, cuprous chloride, bromide or iodide, where cuprous iodide is preferred, a small amount of water, where about 1 to 4 percent is preferred, in a reaction inert solvent such as 1,2-dimethoxyethane, diglyme, t-butyl methyl ether, tetrahydrofuran, benzene, toluene, where toluene is preferred, from about 40° to 150° C., where about 80° to about 120° C. is preferred. Alternatively, the conversion of 5 to 1b, wherein R³ is an optionally substituted aryl or heteroaryl group, can be accomplished by treating 5 and an aryl or heteroaryl chloride, bromide, iodide, or sulfonate, where the bromide is preferred, with a base such as an alkali metal carbonate, an alkali metal amine base, an alkali metal phosphonate, or an alkali metal alkoxide, where cesium carbonate is preferred, a phosphine ligand, where 9,9-dimethyl-4,5-bis(diphenylphosphino)xanthene (XANTPHOS) is preferred, a palladium species, such as palladium (II) acetate or tris(dibenzylideneacetone)dipalladium (0) or the corresponding chloroform adduct, where tris(dibenzylideneacetone)dipalladium (0) is preferred, in an inert solvent such as 1,4-dioxane or toluene, where 1,4-dioxane is preferred, at a temperature from about 40° to about 160° C., where 80° to 120° C. is preferred. Conversion of 1b to 1a can be achieved by placing 1b in a reaction inert solvent such as a lower alcohol, wherein methanol or ethanol are preferred, adding a noble metal catalyst suspended on a solid support, such as platinum or palladium, where 10% palladium on carbon is preferred, then placing the mixture under a hydrogen atmosphere, from about 1 atm to 5 atm, where about 3 to 4 atm is preferred, at a temperature from about 10° to about 100° C., where 40° to 60° C. is preferred, and then shaking the mixture. In the case where R⁶=benzyl or some other group that is labile towards hydrogenation conditions, the corresponding secondary amine derivative (R⁶=H) is formed. If R⁶=H, further functionalization of the secondary amine can be carried out under standard alkylation or reductive amination conditions known to one skilled in the art.

Another route to 1b is shown in Scheme 1. The conversion of 7 to 8, wherein R³ is Ar¹, an optionally substituted aryl or heteroaryl group as described above and in claim 1, can be accomplished by treating 7, an aryl or heteroaryl chloride, bromide, iodide, or sulfonate, where the bromide is preferred, with a base such as potassium phosphate, potassium carbonate, sodium carbonate, thallium carbonate, cesium carbonate, potassium tert-butoxide, lithium tert-butoxide, or sodium tertbutoxide, where potassium carbonate is preferred, a diamine, such as 1,2-ethylenediamine, N,N′-dimethyl-ethylenediamine, or cis-1,2-diaminocyclohexane, where N,N′-dimethylethylenediamine is preferred, cuprous chloride, bromide or iodide, where cuprous iodide is preferred, and a small amount of water, where about 1-4% is preferred, in a reaction inert solvent such as 1,2-dimethoxyethane, diglyme, t-butyl methyl ether, tetrahydrofuran, benzene, toluene, where toluene is preferred, from about 40° to about 150° C., where about 80° to 120° C. is preferred. Alternatively, the conversion of 7 to 8 can be accomplished by treating 7 and an aryl or heteroaryl chloride, bromide, iodide, or sulfonate, where the bromide is preferred, with a base such as an alkali metal carbonate, an alkali metal amine base, an alkali metal phosphonate, or an alkali metal alkoxide, where cesium carbonate is preferred, a phosphine ligand, where 9,9-dimethyl-4,5-bis(diphenylphosphino)xanthene (XANTPHOS) is preferred, a palladium species, such, as palladium (II) acetate or tris (dibenzylideneacetone)dipalladium (0) or the corresponding chloroform adduct, where tris(dibenzylideneacetone)dipalladium (0) is preferred, in an inert solvent such as 1,4-dioxane or toluene, where 1,4-dioxane is preferred, at a temperature from about 40° to 160° C., where 80° to 120° C. is preferred.

Compound 8 can also be prepared by condensation of R³—NH₂ with 8a, in a solvent such as water, acetonitrile, 1,4-dioxane, or tetrahydrofuran, where tetrahydrofuran is preferred, at a temperature from about 100 to 120° C., where 50° to 80° C. is preferred, in the presence or absence of a base, such as triethylamine, diisopropylethyl amine, an alkali metal hydroxide or an alkali metal carbonate, where cesium carbonate is preferred, where the group B of 8a can be F, Cl, Br, I, OC₁-C₄ alkyl, OH, or an activated carboxylic acid group derived from reaction of the acid with a standard carboxylic acid activating reagent such as, but not limited to, a carbodiimide (dicyclohexyl carbodiimide, commonly abbreviated “DCC,” 1-(3-dimethylaminopropyl)-3-ethylcarbodiimide hydro-chloride salt) or tripropyl-phosphonic anhydride, where B=Cl is preferred, where the group A of 8a can be F, Cl, Br, I, or an alkyl or aryl sulfonate, where A=Cl is preferred. Synthesis of 1b can be accomplished by reacting 4 and 8 in a solvent such as tetrahydrofuran, tert-butyl methyl ether, or 1,4-dioxane, where tetrahydrofuran is preferred, with an alkali metal amine base, such as sodium bis(trimethylsilylamide), potassium bis(trimethylsilylamide), lithium bis(trimethyl-silylamide), or lithium diisopropylamide, or an alkali metal hydride, such as sodium hydride or potassium hydride, where sodium bis(hexamethylsilylamide) is preferred, followed by the optional addition of diethylchlorophosphonate (in which case lithium diisopropyl amide is the preferred base) from about −30° to about 100° C., preferably from −10° to 30° C. Compound 1b can then be converted to compound 1a as described above. In the case where R⁶=benzyl or some other group that is labile towards hydrogenation conditions, the corresponding NH derivative (R⁶=H) is formed. If R⁶=H, further functionalization of the secondary amine can be carried out under standard alkylation or reductive amination conditions known to one skilled in the art.

Another method to make compounds of formula 1b described in Scheme 1 starts from pyridylaldehyde 2b, where D=chloro or fluoro, where fluoro is preferred. Reacting 2b and 8 in a solvent such as tetrahydrofuran, tert-butylmethyl ether, or 1,4-dioxane, where tetrahydrofuran is preferred, with an alkali metal amine base, such as sodium bis(trimethylsilylamide), potassium bis(trimethylsilylamide), lithium bis-(trimethyl-silylamide), or lithium diisopropylamide, or an alkali metal hydride, such as sodium hydride or potassium hydride, where sodium bis(hexamethylsilylamide) is preferred, followed by the optional addition of diethylchlorophosphonate (in which case lithium diisopropyl amide is the preferred base) from about −30° to 100° C., preferably from −10° to 30° C., affords F. F can then be converted to 1b and 1b can be converted to 1a as described above.

Scheme 2 illustrates general methods suitable for preparing compounds of formula I wherein X is O (Formula 1e below).

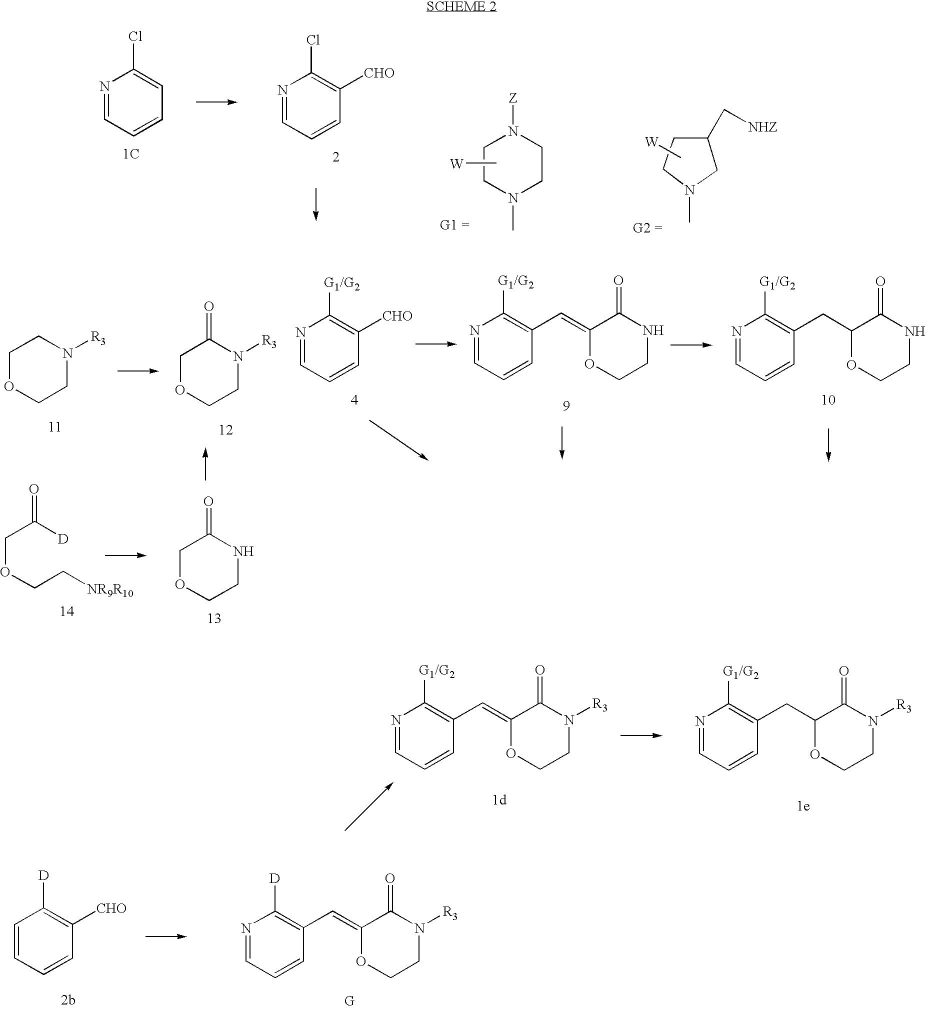

Treatment of a mixture of 3-fluoro-pyridine-2-carbaldehyde 2 and G1* or G2* in a solvent such as water, 1,4-dioxane, n-butanol, N,N-dimethylformamide, dimethyl sulfoxide, or acetonitrile, where water is preferred, with a base that is inert toward 2, G1 or G2, and the solvent, such as a trialkyl amine or an alkali metal carbonate, wherein potassium carbonate is preferred, at reaction temperature from about 40° to about 150° C., where 90° to 120° C. is preferred, affords pyridyl piperazinyl aldehyde 4. Addition of 4 and an N-substituted morpholinone 12, where the N-substituent is vinyl or C(═O)R, (wherein R=C₁-C₈ alkyl, straight chain or branched, C₃-C₈ cycloalkyl, or aryl), wherein C(═O)R with R=tertbutyl is preferred (Sasaki, H. et al. J. Med. Chem., 1991, 34, 628-633), with an amine or hydride metal base such as sodium hydride or sodium bis(trimethylsilylamide), where sodium bis(trimethylsilylamide) is preferred, in an inert reaction solvent, where tetrahydrofuran is preferred, from about −30° to about 100° C., preferably from about −10° to about 30° C., affords 9. Reduction of the carbon-carbon double bond of 9 to generate 10 can be achieved by placing 9 in a reaction inert solvent such as a lower alcohol, wherein methanol or ethanol are preferred, adding a noble metal catalyst suspended on a solid support, such as platinum or palladium, where 10% palladium on carbon is preferred, then placing the mixture under a hydrogen atmosphere, from about 1 atm to 5 atm, where about 3 to 4 atm is preferred, at a temperature from about 10° to about 100° C., where 40 to 60° C. is preferred, and then shaking the mixture. In the case where R⁶=benzyl or some other group that is labile towards hydrogenation conditions, the corresponding NH derivative (R⁶=H) is formed.

The conversion of 10 to 1e, wherein R³ is an optionally substituted aryl or heteroaryl group, can be accomplished by treating 10, an aryl or heteroaryl chloride, bromide, iodide, or sulfonate, where the bromide is preferred, a base such as potassium phosphate, potassium carbonate, sodium carbonate, thallium carbonate, cesium carbonate, potassium tert-butoxide, lithium tertbutoxide, or sodium tert-butoxide, where potassium carbonate is preferred, a diamine, such as 1,2-ethylenediamine, N,N′-dimethylethylenediamine, or cis-1,2-diaminocyclo-hexane, where N,N′-dimethylethylenediamine is preferred, a cuprous chloride, bromide or iodide, where cuprous iodide is preferred, a small amount of water, where about 1 to 4 percent is preferred, in a reaction inert solvent such as 1,2-dimethoxyethane, diglyme, t-butyl methyl ether, tetrahydrofuran, benzene, toluene, where toluene is preferred, from about 40° to 150° C., where about 80° to about 120° C. is preferred. Alternatively, the conversion of 10 to 1e, wherein R³ is an optionally substituted aryl or heteroaryl group, can be accomplished by treating 10 and an aryl or heteroaryl chloride, bromide, iodide, or sulfonate, where the bromide is preferred, with a base such as an alkali metal carbonate, an alkali metal amine base, an alkali metal phosphonate, or an alkali metal alkoxide, where cesium carbonate is preferred, a phosphine ligand, where 9,9-dimethyl-4,5-bis(diphenylphosphino)xanthene (XANTPHOS) is preferred, a palladium species, such as palladium (II) acetate or tris(dibenzylideneacetone)dipalladium (0) or the corresponding chloroform adduct, where tris(dibenzylideneacetone)dipalladium (0) is preferred, in an inert solvent such as 1,4-dioxane or toluene, where 1,4-dioxane is preferred, at a temperature from about 40° to about 160° C., where 80° to 120° C. is preferred. If R⁶=H, then further functionalization of the secondary amine can be carried out under standard alkylation or reductive amination conditions known to one skilled in the art.

Another route to access compounds of formula 1d and 1e is shown in Scheme 2. The conversion of 9 to 1d, wherein R3 is an optionally substituted aryl or heteroaryl group, can be accomplished by treating 9, an aryl or heteroaryl chloride, bromide, iodide, or sulfonate, where the bromide is preferred, a base such as potassium phosphate, potassium carbonate, sodium carbonate, thallium carbonate, cesium carbonate, potassium tert-butoxide, lithium tert-butoxide, or sodium tert-butoxide, where potassium carbonate is preferred, a diamine, such as 1,2-ethylenediamine, N,N′-dimethyl-ethylenediamine, or cis-1,2-diaminocyclohexane, where N,N′-dimethylethylenediamine is preferred, cuprous chloride, bromide or iodide, where cuprous iodide is preferred, a small amount of water, where about 1 to 4 percent is preferred, in a reaction inert solvent such as 1,2-dimethoxyethane, diglyme, t-butyl methyl ether, tetrahydrofuran, benzene, toluene, where toluene is preferred, from about 40° to about 150° C., where about 80° to 120° C. is preferred. Alternatively, the conversion of 9 to 1d, wherein R³ is an optionally substituted aryl or heteroaryl group, can be accomplished by treating 9 and an aryl or heteroaryl chloride, bromide, iodide, or sulfonate, where the bromide is preferred, with a base such as an alkali metal carbonate, an alkali metal amine base, an alkali metal phosphonate, or an alkali metal alkoxide, where cesium carbonate is preferred, a phosphine ligand, where 9,9-dimethyl-4,5-bis(diphenylphosphino)xanthene (XANTPHOS) is preferred, a palladium species, such as palladium(II)acetate or tris(dibenzylideneacetone) dipalladium(0) or the corresponding chloroform adduct, where tris(dibenzylidene-acetone)dipalladium(0) is preferred, in an inert solvent such as 1,4-dioxane or toluene, where 1,4-dioxane is preferred, at a temperature from about 40° to about 160° C., where 80° to 120° C. is preferred. Conversion of 1d to 1e can be achieved by placing 1d in a reaction inert solvent such as a lower alcohol, wherein methanol or ethanol are preferred, adding a noble metal catalyst suspended on a solid support, such as platinum or palladium, where 10% palladium on carbon is preferred, then placing the mixture under a hydrogen atmosphere, from about 1 atm to 5 atm, where about 3 to 4 atm is preferred, at a temperature from about 10° to about 100° C., where 40° to 60° C. is preferred, and then shaking the mixture. In the case where R⁶=benzyl or some other group that is labile towards hydrogenation conditions, the corresponding secondary amine derivative (R⁶=H) is formed. If R⁶=H, further functionalization of the secondary amine can be carried out under standard alkylation or reductive amination conditions known to one skilled in the art.

Another route that allows for the access to 1d is shown in Scheme 2. The conversion of 13 to 12, wherein R³ is an optionally substituted aryl or heteroaryl group, can be accomplished by treating 13, an aryl or heteroaryl chloride, bromide, iodide, or sulfonate, where the bromide is preferred, a base such as potassium phosphate, potassium carbonate, sodium carbonate, thallium carbonate, cesium carbonate, potassium tert-butoxide, lithium tertbutoxide, or sodium tert-butoxide, where potassium carbonate is preferred, a diamine, such as 1,2-ethylenediamine, N,N′-dimethyl-ethylenediamine, or cis-1,2-diamino-cyclohexane, where N,N′-dimethylethylenediamine is preferred, cuprous chloride, bromide or iodide, where cuprous iodide is preferred, a small amount of water, where about 1 to 4 percent is preferred, in a reaction inert solvent such as 1,2-dimethoxyethane, diglyme, t-butyl methyl ether, tetrahydrofuran, benzene, toluene, where toluene is preferred, from about 40° to about 150° C., where about 80° to 120° C. is preferred affords 12. Alternatively, the conversion of 13 to 12, wherein R³ is an optionally substituted aryl or heteroaryl group, can be accomplished by treating 13 and an aryl or heteroaryl chloride, bromide, iodide, or sulfonate, where the bromide is preferred, with a base such as an alkali metal carbonate, an alkali metal amine base, an alkali metal phosphonate, or an alkali metal alkoxide, where cesium carbonate is preferred, a phosphine ligand, where 9,9-dimethyl-4,5-bis(diphenylphosphino)xanthene (XANTPHOS) is preferred, a palladium species, such as palladium(II)acetate or tris(dibenzylideneacetone)dipalladium(0) or the corresponding chloroform adduct, where tris(dibenzylideneacetone)dipalladium(0) is preferred, in an inert solvent such as 1,4-dioxane or toluene, where 1,4-dioxane is preferred, at a temperature from about 40° to about 160° C., where 80° to 120° C. is preferred. In addition, 12 is an N-substituted morpholinone, where the R³ group may also be defined as an N-substituent defined as vinyl or C(═O)R, (wherein R=C₁-C₈ alkyl, straight chain or branched, C₃-C₈ cycloalkyl, or aryl), wherein C(═O)R with R=tert-butyl is preferred is prepared by adding RCOCl (where R is defined above) to morpholinone 13 and a tertiary amine base, wherein triethylamine is preferred, in a chlorinated solvent, wherein methylene chloride is preferred at a temperature from −30° C. to 50° C. wherein 0° C. is preferred to afford morpholinone 12.

In turn, morpholinone 13 was prepared using literature methods (Pfeil, E., et al., Angew. Chem., 1967, 79, 188; Lehn, J.-M., et al., Helv. Chim. Acta, 1976, 59, 1566-1583; Sandmann, G., et al., J. Agric. Food Chem., 2001, 49, 138-141. 13 may also be prepared by condensation of 14 in a solvent such as water, acetonitrile, 1,4-dioxane, or tetrahydrofuran, where tetrahydrofuran is preferred, at a temperature from about 10° to about 120° C., where 50° to 80° C. is preferred, in the presence or absence of a base, such as triethylamine, diisopropylethyl amine, an alkali metal hydroxide or an alkali metal carbonate, where cesium carbonate is preferred, where the group D of 14 can be F, Cl, Br, I, OC1-C4 alkyl, OH, or an activated carboxylic acid group derived from reaction of the acid with a standard carboxylic acid activating reagent such as, but not limited to, a carbodiimide (dicyclohexyl carbodiimide, 1-(3-dimethylaminopropyl)₃-ethyl-carbo-diimide hydrochloride salt) or tripropylphosphonic anhydride, where D=Cl is preferred. R⁹ and/or R¹⁰ can be hydrogen, or an appropriately designed group known in the art which may be removed prior to cyclization such as a carbamate or phthalimide in which the phthalimide is preferred and removed prior to cyclization with hydrazine. Synthesis of 1d can be accomplished by reacting 4 and 12 in a solvent such as tetrahydrofuran, tert-butyl methyl ether, or 1,4-dioxane, where tetrahydrofuran is preferred, with an alkali metal amine base, such as sodium bis(trimethylsilylamide), potassium bis(trimethyl-silylamide), lithium bis(trimethylsilylamide), or lithium diisopropylamide, or an alkali metal hydride, such as sodium hydride or potassium hydride, where sodium bis(hexamethylsilylamide) is preferred, followed by the optional addition of diethylchlorophosphonate (in which case lithium diisopropyl amide is the preferred base) from about −30° to 100° C., preferably from −10° to 30° C. 1d can then be converted to 1e as described above. In the case where R⁶=benzyl or some other group that is labile towards hydrogenation conditions, the corresponding NH derivative (R⁶=H) is formed. If R⁶=H, further functionalization of the secondary amine can be carried out under standard alkylation or reductive amination conditions known to one skilled in the art.

Alternatively, 12 can also be prepared by treatment of 11 with an appropriate oxidation reagent such as potassium permanganate and a quaternary ammonium salt where benzyltrimethylammonium chloride is preferred in a chlorinated solvent such as methylene chloride, dichloroethane, chloroform, where methylene chloride is preferred, at a temperature from about 25° to 160° C., where 30° to 60° C. is preferred. The synthesis of 11 can be accomplished by treating morpholine with an aryl or heteroaryl chloride bromide, iodide, or sulfonate, where the bromide is preferred, a base such as potassium phosphate, potassium carbonate, sodium carbonate, thallium carbonate, cesium carbonate, potassium tert-butoxide, lithium tert-butoxide, or sodium tert-butoxide, where sodium tert-butoxide is preferred, a phosphine ligand, where BINAP or triphenylphosphine is preferred, a palladium species, such as palladium(II)acetate or tris(dibenzylideneacetone)dipalladium(0) or the corresponding chloroform adduct, where tris(dibenzylideneacetone)dipalladium(0) is preferred, in an inert solvent such as 1,4-dioxane or toluene, where 1,4-dioxane is preferred, at a temperature from about 40° to about 160° C., where 80° to 120° C. is preferred.

Another method for synthesizing compounds of formula 1d described in Scheme 2 starts from pyridylaldehyde 2b, where D=chloro or fluoro, where fluoro is preferred. Reacting 2b and 12 in a solvent such as tetrahydrofuran, tert-butyl methyl ether, or 1,4-dioxane, where tetrahydrofuran is preferred, with an alkali metal amine base, such as sodium bis(trimethylsilylamide), potassium bis(trimethylsilylamide), lithium bis (trimethyl-silylamide), or lithium diisopropylamide, or an alkali metal hydride, such as sodium hydride or potassium hydride, where sodium bis(hexamethylsilylamide) is preferred, followed by the optional addition of diethylchlorophosphonate (in which case lithium diisopropyl amide is the preferred base) from about −30° to about 100° C., preferably from −10° to 30° C., affords 1d. 1d can then be converted to 1e as described above. The aryl halides used in the coupling are prepared via the general methods outlined in U.S. Pat. No. 5,612,359 (Preparations 2-9); Guay, D., et al. Biorg. Med. Chem. Lett. 2002, 12,1457-1461; Sall, D. J., et al. J. Med. Chem. 2000, 43, 649-663; Olah, G. A., et al. J. Am. Chem. Soc. 1971, 93, 6877-6887; Brown, H. C. et al. J. Am. Chem. Soc. 1957, 79, 1906-1909; Nenitzescu, C., et al., I. J. Am. Chem. Soc. 1950, 72, 3483-3486; Muci, A. R.; Buchwald, S. L. Top. Curr. Chem.; Springer-Verlag: Berlin Heidelberg, 2002; 219,131-209; DE 19650708; Howard, H. R.; EP 104860; EP 0501579A; Wang, X., et al. Tetrahedron Lett., 2000, 41, pp. 4335-4338. In cases where an alcohol was present on the aryl halide, treatment of the alcohol with an alkali metal hydride or alkali metal hydroxide, such as sodium hydride, potassium hydride, sodium hydroxide, potassium hydroxide, or cesium hydroxide, where sodium hydride is preferred, in a solvent such as tetrahydrofuran, N,N-dimethylformamide, or dimethylsulfoxide, where tetrahydrofuran is preferred, at a temperature from about −20° to about 50° C., followed by addition of an alkyl halide or tosylate, where an alkyl iodide is preferred, affords the corresponding ether.

Examples of specific compounds of Formula 1 are the following:

### EXAMPLE 1

### 1-[4-(3,5-Dimethyl-isoxazol-4-yl)-phenyl]-3-[2-(4-methyl-piperazin-1-yl)-pyridin-3-ylmeth-ylene]-piperidin-2-one

### EXAMPLE 2

### 2-[2-(4-Methyl-piperazin-1-yl)-pyridin-3-ylmethylene]-4-[4-(tetrahydro-pyran-4-yl)-phen-yl]-morpholin-3-one

### EXAMPLE 3

### 1-[4-(3,5-Dimethyl-isoxazol-4-yl)-phenyl]-3-[2-(4-methyl-piperazin-1-yl)-pyridin-3-ylmeth-yl]-piperidin-2-one

### EXAMPLE 4

### 2-[2-(4-Methyl-piperazin-1-yl)-pyridin-3-ylmethyl]-4-[4-(tetrahydro-pyran-4-yl)-phenyl]-morpholin-3-one

### EXAMPLE 5

### 1-[4-(2-Methyl-oxazol-4-yl)-phenyl]-3-[2-(4-methyl-piperazin-1-yl)-pyridin-3-ylmethyl]-piperidin-2-one

### EXAMPLE 6

### 1-[4-(2-tert-Butyl-oxazol-4-yl)-phenyl]-3-[2-(4-methyl-piperazin-1-yl)-pyridin-3-ylmethyl]-piperidin-2-one

### EXAMPLE 7

### 1-[4-(2-Isopropyl-oxazol-4-yl)-phenyl]-3-[2-(4-methyl-piperazin-1-yl)-pyridin-3-ylmethyl]-piperidin-2-one

### EXAMPLE 8

### 1-[4-(2,5-Dimethyl-oxazol-4-yl)-phenyl]3-[2-(4-methyl-piperazin-1-yl)-pyridin-3-ylmeth-yl]-piperidin-2-one

### EXAMPLE 9

### 3-[2-(4-Methyl-piperazin-1-yl)-pyridin-3-ylmethyl]-1-[4-(tetrahydro-pyran-4-yl)-phen-yl]-piperidin-2-one

### EXAMPLE 10

### 3-[2-(4-Methyl-piperazin-1-yl)-pyridin-3-ylmethyl]-1-[4-(tetrahydro-pyran-4-yl)-phenyl]-pyrrolidin-2-one

### EXAMPLE 11

### 3-[2-(4-Methyl-piperazin-1-yl)-pyridin-3-ylmethyl]-1-(4-oxazol-2-yl-phenyl)-pyrrolidin-2-one

### EXAMPLE 12

### [2-(4-Methyl-piperazin-1-yl)-pyridin-3-ylmethyl]-1-(4-oxazol-4-yl-phenyl)-pyrrolidin-2-one

### EXAMPLE 13

### 3-[2-(4-Methyl-piperazin-1-yl)-pyridin-3-ylmethyl]-1-(4-oxazol-5-yl-phenyl)-pyrrolidin-2-one

### EXAMPLE 14

### 1-[4-(2-Methyl-oxazol-4-yl)-phenyl]-3-[2-(4-methyl-piperazin-1-yl)-pyridin-3-ylmethyl]-pyrrolidin-2-one

### EXAMPLE 15

### 1-[4-(1-Methoxy-cyclobutyl)-phenyl]-3-[2-(4-methyl-piperazin-1-yl)-pyridin-3-ylmethyl]-piperidin-2-one

### EXAMPLE 16

### 3-[2-(4-Methyl-piperazin-1-yl)-pyridin-3-ylmethyl]-1-(4-trifluoromethyl-phenyl)-pyrrolidin-2-one

### EXAMPLE 17

### 1-[4-(1-Hydroxy-cyclopentyl)phenyl]-3-[2-(4-methyl-piperazin-1-yl)-pyridin-3-ylmethyl]-pyrrolidin-2-one

### EXAMPLE 18

### 1-[4-(1-Hydroxy-1-methyl-ethyl)-phenyl]-3-[2-(4-methyl-piperazin-1-yl)-pyridin-3-ylmethyl]-pyrrolidin-2-one

### EXAMPLE 19

### 1-(4-tert-Butyl-phenyl)-3-[2-(4-methyl-piperazin-1-yl)-pyridin-3-ylmethyl]-pyrrolidin-2-one

### EXAMPLE 20

### 1-[4-(1-Hydroxy-cyclopentyl)-phenyl]-3-[2-(4-methyl-piperazin-1-yl)-pyridin-3-ylmethyl]-piperidin-2-one

### EXAMPLE 21

### 1-(4-tert-Butyl-phenyl)-3-[2-(4-methyl-piperazin-1-yl)-pyridin-3-ylmethyl]-piperidin-2-one

### EXAMPLE 22

### 3-[2-(4-Methyl-piperazin-1-yl)-pyridin-3-ylmethyl]-1-phenyl-piperidin-2-one

### EXAMPLE 23

### 3-[2-(4-Methyl-piperazin-1-yl)-pyridin-3-ylmethyl]-1-(4-trifluoromethoxy-phenyl)-piperidin-2-one

### EXAMPLE 24

### 11-[4-(1-Hydroxy-1-methyl-ethyl)-phenyl]-3-[2-(4-methyl-piperazin-1-yl)-pyridin-3-yl-methyl]-piperidin-2-one

### EXAMPLE 25

### 1-[4-(1-Hydroxy-cyclobutyl)-phenyl]-3-[2-(4-methyl-piperazin-1-yl)-pyridin-3-ylmethyl]-piperidin-2-one

Preparation 1

2-(4-Methyl-piperazin-1-yl)-pyridine-3-carbaldehyde. A mixture of 1-methylpiperazine (12.8 mL, 120 mmol), potassium carbonate (13.6 g, 99 mmol), and 2-chloro-pyridine-3-carbaldehyde (9.3 g, 66 mmol) in water (75 mL) and 1,4-dioxane (33 mL) was heated at 100° C. for 18 h. The solution was cooled to room temperature, poured into water and extracted with methylene chloride. The combined organic layers were dried (Na₂SO₄) and concentrated to afford 13.2 g of an oil (98% yield). MS (AP/CI) 206.2 (M+1). ¹³C NMR (100 MHz, CDCl₃) 46.3, 51.2, 55.2, 116.1, 119.6, 140.6, 152.7, 161.8, 190.1.

Preparation 2: General Aldol Procedure

3-[2-(4-Methyl-piperazin-1-yl)-pyridin-3-ylmethylene]-pyrrolidin-2-one. A solution of 10.0 g (49 mmol) of 2-(4-methyl-piperazin-1-yl)-pyridine-3-carbaldehyde and 6.2 g (49 mmol) of N-acetylpyrrolidinone in 100 mL of tetrahydro-furan was slowly added to a 0° C. suspension of 6.45 g (161 mmol, 60% by weight) of sodium hydride in 100 mL of tetrahydrofuran over a 30 minute period. After the addition was complete, the mixture was stirred 10 min at 0° C. and then stirred at room temperature for 18 h. The reaction mixture was quenched into water and extracted with methylene chloride. The organic layer was dried with sodium sulfate and concentrated to provide a yellow solid. Recrystallization from ethyl acetate provided 4.9 g (37% yield) of the title compound as a white solid. MS (AP/CI) 273.3 (M+H). ¹³C NMR (100 MHz, CDCl₃) 26.3, 39.9, 46.3, 50.4, 55.3, 116.7, 121.7, 127.2, 130.8, 137.0, 147.7, 161.3, 172.6.

Preparation 3

3-[2-(4-Methyl-piperazin-1-yl)-pyridin-3-ylmethylene]-piperidin-2-one. The title compound was prepared in a procedure analogous to that described in Preparation 2. MS (AP/CI) 287.3 (M+H). ¹³C NMR (100 MHz, CDCl₃) 23.2, 26.6, 42.5, 46.3, 50.0, 55.4, 116.1, 121.4, 129.0, 133.4, 138.4, 147.6, 161.0, 166.5.

Preparation 4

1-[4-(3,5-Dimethyl-isoxazol-4-yl)phenyl]-3-[2-(4-methyl-piperazin-1-yl)-pyridin-3-ylmeth-ylene]-piperidin-2-one. The title compound was prepared in a procedure analogous to that described in Preparation 2. MS (AP/CI) 458.2 (M+H). ¹³C NMR (100 MHz, CDCl₃) 11.0, 11.8, 23.6, 27.1, 46.3, 49.9, 51.5, 55.4, 116.1, 116.3, 121.6, 126.6, 128.8, 129.3, 129.8, 134.0, 138.2, 143.1, 147.6, 158.8, 161.0, 164.9, 165.5.

Preparation 5: General Aldol Procedure for Morpholinones

2-[2-(4-Methyl-piperazin-1-yl)-pyridin-3-ylmethylene]-4-[4-(tetrahydro-pyran-4-yl)-phen-yl]-morpholin-3-one. A solution of of 2-(4-methyl-piperazin-1-yl)pyridine-3-carbaldehyde (196 mg, 0.96 mmol) and 4-[4-(tetrahydro-pyran-4-yl)-phenyl]-morpholin-3-one (300 mg, 1.1 mmol) in 10 ml tetrahydrofuran was added to a suspension of 115 mg of NaH (2.9 mmol, 60% by weight) in 5 ml tetrahydrofuran. The resulting mixture was heated at 65° C. for 18 h. After quenching into water, the mixture was extracted three times with dichloromethane. The combined organic extracts were dried with Na₂SO₄ and concentrated to an oil. Recrystallization from ether afforded 240 mg of the title compound as a tan solid (56% yield). MS (AP/CI) 449.3 (M+H). ¹³C NMR (100 MHz, CDCl₃) 34.1, 41.4, 46.3, 49.3, 50.6, 55.5, 64.6, 68.5, 110.1, 117.1, 120.6, 125.3, 127.8, 138.4, 140.1, 144.8, 147.0, 159.8, 161.2.

Preparation 6: General Hydrogenation Procedure

3-[2-(4-Methyl-piperazin-1-yl)-pyridin-3-ylmethyl]-pyrrolidin-2-one. To a solution of 4.4 g (16.1 mmol) of 3-[2-(4-methyl-piperazin-1-yl)-pyridin-3-ylmethylene]-pyrrolidin-2-one in 200 mL of ethanol was added 1.1 g of 10% Pd/C. Hydrogenation at 45 psi with heating at 50° C. was complete after 24 h. The reaction was filtered over celite using ethanol and concentrated to 4.4 g (99% yield) of the title compound as an oil. MS (AP/CI) 275.3 (M+H). ¹³C NMR (100 MHz, CDCl₃) 27.4, 32.3, 40.6, 41.4, 46.4, 50.6, 55.6, 118.8, 127.4, 138.5, 146.2, 162.3, 180.2.

Preparation 7

3-[2-(4-Methyl-piperazin-1-yl)-pyridin-3-ylmethyl]-piperidin-2-one. The title compound was prepared in a procedure analogous to that described in Preparation 6. MS (AP/CI) 289.3 (M+H). ¹³C NMR (100 MHz, CDCl₃) 21.3, 25.4, 32.8, 41.2, 42.3, 46.1, 50.4, 55.4, 118.8, 127.6, 138.7, 145.9, 162.2, 174.9.

Preparation 8

1-[4-(3,5-Dimethyl-isoxazol-4-yl)phenyl]-3-[2-(4-methyl-piperazin-1-yl)-pyridin-3-ylmeth-yl]-piperidin-2-one. The title compound was prepared in a procedure analogous to that described in Preparation 6. MS (AP/CI) 460.3 (M+H). ¹³C NMR (100 MHz, CDCl₃) 11.1, 11.8, 22.3, 26.2, 33.8, 42.2, 46.4, 50.6, 51.7, 55.7, 116.3, 118.8, 126.7, 127.6, 128.9, 129.9, 139.1, 142.9, 146.2, 158.9, 162.3, 165.6, 172.7.

Preparation 9: General Hydrogenation Procedure for Morpholinones

2-[2-(4-Methyl-piperazin-1-yl)-pyridin-3-ylmethyl]-4-[4-(tetrahydro-pyran-4-yl)-phenyl]-morpholin-3-one. To a solution of 2-[2-(4-methyl-piperazin-1-yl)-pyridin-3-ylmethylene]-4-[4-(tetrahydro-pyran-4-yl)-phenyl]-morpholin-3-one (140 mg, 0.31 mmol) in 40 mL of ethanol was added 140 mg of 10% Pd/C. After hydrogenation at 40 psi for 18 h, additional 10% Pd/C (140 mg) was added. Hydrogenation at 40 psi was complete after another 18 h. The mixture was filtered over Celite using ethanol and concentrated to an oil. Purification by silica gel flash column chromatography (88:12, dichloromethane: methanol) afforded 25 mg of the title compound as an oil (18% yield). MS (AP/CI) 451.3 (M+H). ¹³C NMR (100 MHz, CDCl₃) 33.9, 34.1, 41.4, 46.3, 50.5, 50.6, 55.6, 62.9, 68.5, 77.6, 118.6, 125.9, 126.1, 127.9, 139.2, 140.0, 145.1, 146.4, 162.1, 168.9.

Preparation 10

### 4-[4-(Tetrahydro-pyran-4-yl)-phenyl]-morpholin-3-one

Step 1: 4-[4-(Tetrahydro-pyran-4-yl)-Phenyl]-morpholine. The title compound was prepared in a procedure analogous to that described in Buchwald et al. MS (APCI) 248.2 (M+H). Diagnostic ¹³C NMR (100 MHz, CDCl₃) 34.3, 40.8, 49.8, 67.2, 68.7, 116.1, 127.6.

Step 2: 4-[4-(Tetrahydro-pyran-4-yl)-phenyl]-morpholin-3-one. 4-[4-(Tetrahydro-pyran-4-yl)-phenyl]-morpholine (2.37 g, 9.6 mmol), potassium permanganate (4.54 g, 29 mmol) and benzyltriethylammonium chloride (6.59 g, 29 mmol) were combined in dichloromethane (60 ml). After heating 4 h at 45° C., the cooled reaction mixture was quenched with aqueous sodium bisulfite and extracted three times with dichloromethane. The combined organic extracts were dried (Na₂SO₄) and concentrated to an oil. Purification by silica gel chromatography afforded the title compound as a white foam (600 mg, 24% yield). MS (APCI) 262.2 (M+H). ¹³C NMR (100 MHz, CDCl₃) 34.0, 41.4, 49.9, 64.3, 68.5, 68.8, 125.8, 127.9, 139.7, 145.0, 166.9.

Preparation 11

1-[4-(3,5-Dimethyl-isoxazol-4-yl)phenyl]-Piperidin-2-one. 1-(4-Iodo-phenyl)-piperidin-2-one (1.1 g, 3.7 mmol), potassium phosphate (1.57 g, 7.4 mmol), tetrakis (triphenylphosphine)palladium (0) (214 mg, 0.19 mmol) and 3,5-dimethyloxazole-4-boronic acid (780 mg, 5.5 mmol) were combined in 25 mL dioxane. After heating at 90° C. for 18 h, the cooled reaction mixture was poured in aqueous sodium bicarbonate and extracted with dichloromethane. The combined organic extracts were dried (Na₂SO₄) and concentrated to an oil. Purification by silica gel chromatography (4:96, methanol:dichloromethane) afforded 340 mg of the title compound as an oil (34% yield). ¹H NMR (400 MHz, CDCl₃) □1.88-1.94 (m, 4H), 2.23 (s, 3H), 2.36 (s, 3H), 2.53 (t, 2H, J=6.2 Hz), 3.62-3.65 (m, 2H), 7.22 (d, 2H, J=8.4 Hz), and 7.29 (d, 2H, J=8.4 Hz). MS (APCI) 271.2 (M+1).

Aryl Halides

In cases where an alcohol was present on the aryl halide, treatment of the alcohol with an alkali metal hydride or alkali metal hydroxide, such as sodium hydride, potassium hydride, sodium hydroxide, potassium hydroxide, or cesium hydroxide, where sodium hydride is preferred, in a solvent such as tetrahydrofuran, N,N-dimethyl-formamide, or dimethylsulfoxide, where tetrahydrofuran is preferred, at a temperature from about −20° to about 50° C., followed by addition of an alkyl halide or tosylate, where an alkyl iodide is preferred, affords the corresponding ether.

Preparation 12

2-(4-Bromo-phenyl)-propan-2-ol. A solution of methyl p-bromobenzoate (3 g, 13.2 mmol) in tetrahydrofuran (14 mL) cooled to −30° C. was treated dropwise with methyl magnesium bromide (1 M in diethyl ether, 105.5 mmol, 105.5 mL). Upon completion of addition, the resulting suspension was allowed to warm to room temperature and was stirred for 5 h. Saturated aqueous ammonium chloride (100 mL) was added slowly and the mixture was diluted with ethyl acetate (100 mL). The organic and aqueous layers were separated and the aqueous layer was extracted with ethyl acetate (3×50 mL). The combined organic layers were dried over magnesium sulfate, were filtered, and the solvent was removed in vacuo. Purification by silica gel chromatography (10:1 hexanes—ethyl acetate) gave 2.2 g (79% yield) of 2-(4-bromo-phenyl)-propan-2-ol. ¹³C NMR (100 MHz, CDCl₃) d 148.4, 131.4, 126.6, 120.8, 72.5, 31.9; MS (AP/CI) 197.1, 199.1 (M+H)+.

Preparation 13

2-(5-Bromo-pyridin-2-yl)-propan-2-ol. The title compound was prepared using ethyl-5-bromo-2-carboxypyridine, but otherwise followed the general procedure for Preparation 12. ¹³C NMR (100 MHz, CDCl₃) d 165.1, 148.9, 139.7, 120.4, 118.9, 72.2, 30.7; MS (AP/CI) 216.0, 218.1 (M+H)+.

Preparation 14

1-(4-Bromo-phenyl)-cyclopentanol. The title compound was prepared using the procedure detailed for Preparation 12. ¹H NMR (400 MHz, CDCl₃) d 7.44 (d, J=8.3 Hz, 2H), 7.35 (d, J=8.7 Hz, 2H), 1.9 (m, 6H), 1.8 (m, 2H), 1.75 (s, 1H); ¹³C NMR (100 MHz, CDCl₃) d 146.4, 131.4, 127.2, 120.8, 83.4, 42.2, 24.1.

Preparation 15

1-(4-Bromo-phenyl)-cyclobutanol. The title compound was prepared using the procedure detailed for Preparation 12. ¹³C NMR (400 MHz, CDCl₃) d 145.5, 131.7, 127.1, 121.3, 76.8, 37.2, 13.2; MS (AP/CI) 209.0, 211.0 (M+H−H2O)+.

Preparation 16

4-(4-Bromo-phenyl)-tetrahydro-pyran-4-ol. The title compound was prepared using the procedure detailed for Preparation 12. ¹³C NMR (100 MHz, CDCl₃) d 38.8, 63.9, 70.6, 121.3, 126.6, 131.7, 147.4.

Preparation 17

### 4-(4-Bromophenyl)-tetrahydropyran

A solution of 4-(4-bromo-phenyl)-tetrahydro-pyran-4-ol (859 mg, 3.3 mmol) and triethylsilane (596 μL, 3.7 mmol) in 12 mL dichloromethane was chilled in an ice bath. Trifluoroacetic acid (2.54 mL, 33 mmol) was added in a dropwise manner over 20 min. After 1 h at 0° C. the reaction mixture was stirred at room temperature for 3 h. 1N aqueous NaOH was added until the aqueous pH remained basic, and the mixture was extracted three times with dichloromethane. The organic extracts were combined, dried (Na₂SO₄) and concentrated to an oily solid. Purification by silica gel chromatography (5:95, ethyl acetate:hexanes) afforded the title compound as a white solid (640 mg, 80% yield). ¹³CNMR (100 MHZ, CDCl₃) 34.0, 41.3, 68.5, 120.2, 128.7, 131.8, 145.0.

Preparation 18

1-Bromo-4-(1-methoxy-1-methylethyl)-benzene. 2-(4-Bromo-phenyl)propan-2-ol (Preparation 17, 1.77 g, 8.2 mmol) and methyl iodide (0.5 mL, 8.2 mmol) in tetrahydrofuran (100 mL) were treated with sodium hydride (60% dispersion in mineral oil, 328 mg, 8.2 mmol). The mixture was stirred for 24 h at room temperature, was poured into 0.5 M aqueous hydrochloric acid, and the mixture was extracted with ethyl acetate. The organic layer was washed with brine, was dried over magnesium sulfate, was filtered, and the solvent was removed in vacuo. The residue was purified by silica gel chromatography (200:1 hexanes-ethyl acetate) to afford 500 mg (27% yield) of the title compound. ¹³C NMR (100 MHz, CDCl₃) d 145.4, 131.5, 127.9, 121.0, 76.7, 50.9, 28.1; MS (AP/CI) 197.0, 199.0 (M+H−OMe)+.

Preparation 19

1-Bromo-4-(1-methoxy-cyclobutyl)-benzene. The title compound was prepared using the procedure detailed for Preparation 17. ¹³C NMR (100 MHz, CDCl₃) d 142.5, 131.6, 128.4, 121.4, 81.3, 50.8, 33.0, 13.1; MS (AP/CI) 209.1, 211.1 (M+H−OMe)+.

Preparation 20

5-Bromo-2-ethoxy-pyridine. A solution of freshly prepared sodium ethoxide (sodium, 4.9 g, 210 mmol; absolute ethanol, 100 mL, room temperature) was treated with 2,5-dibromopyridine (10 g, 42 mmol) and was heated at reflux for 18 h. After cooling to room temperature, the mixture was poured into aqueous saturated sodium bicarbonate solution, was extracted with diethyl ether, and the ether layer was washed with brine, was dried over magnesium sulfate, was concentrated in vacuo. Purification by silica gel chromatography (100:1 hexanes-ethyl acetate) gave 7.5 g (88% yield) of the title compound. ¹³C NMR (100 MHz, CDCl₃) d 162.9, 147.7, 141.2, 112.9, 111.6, 62.3, 14.7; MS (AP/CI) 202.1, 204.1 (M+H)+.

Preparation 21

4-(4-Bromo-phenyl)₄-methyl-tetrahydro-pyran. The title compound was prepared in a similar fashion as described in EP0501579A1 ³C NMR (100 MHz, CDCl₃) 29.2, 35.8, 37.7, 37.8, 64.6, 119.9, 127.7, 127.8, 131.7.

Preparation 22

### 3-(4-Bromo-phenyl)₃-methyl-oxetane

Step 1: 2-(4-Bromo-phenyl)-2-methyl-malonic acid diethyl ester

Sodium methoxide (5.96 g, 110.4 mmol) was added to a 0° C. solution of 2-(4-bromo-phenyl)malonic acid diethyl ester (29 g, 92 mmol) in ethanol (200 mL). After 15 min iodomethane (6.9 ml, 110.4 mmol) was added slowly. The reaction mixture was warmed to room temperature and stirred 18 h. Additional portions of iodomethane (1.1 ml, 22 mmol) and sodium methoxide (1.0 g, 22 mmol) were added and the mixture was stirred 66 h. After quenching into water the mixture was extracted three times with ethyl acetate. The combined organic extracts were dried (MgSO₄) and concentrated to provide 16.8 g of the title compound as an oil (55% yield). ¹H NMR (400 MHz, CDCl₃) 1.23-1.25 (m, 6H), 1.83 (s, 3H), 4.19-4.25 (m, 4H), 7.25 (d, 1H, J=7.4 Hz), 7.46 (d, 1H, J=7.4 Hz).

Step 2: 2-(4-Bromo-phenyl)-2-methyl-propane-1,3-diol

A solution of 2-(4-bromo-phenyl)-2-methyl-malonic acid diethyl ester (10 g, 30.3 mmol) in 100 mL diethyl ether was added in a dropwise fashion to a 0° C. solution of 1.0 M lithium aluminium hydride (45 mL, 45 mmol) in 200 mL diethyl ether. After 30 min the reaction was warmed to 40° C. and heated for 4 h. After cooling to 0° C. and quenching with aqueous saturated sodium sulfate, the reaction mixture was filtered through Celite and concentrated to a thick oil. Purification by silica gel chromatography (1:1, ethyl acetate:hexanes) afforded 3.94 g of the title compound (53% yield). ¹³C NMR (100 MHz, CDCl₃) 20.9, 44.3, 69.6, 120.8, 126.8, 128.8, 128.9, 131.8, 142.6.

Step 3: 3-(4-Bromo-phenyl)₃-methyl-oxetane

Triphenylphosphine (3.6 g, 13.8 mmol) was added to a solution of 2-(4-bromo-phenyl)-2-methyl-propane-1,3-diol (1.69 g, 6.89 mmol) in 57 mL toluene. After stirring 5 min, N,N-dimethyldithiacarbonate (3.16 g, 10.34 mmol) and diethyl azodicarboxylate (2.17 mL, 13.79 mmol) were added and the resulting mixture was stirred at room temperature for 18 h. After filtering through Celite the mixture was concentrated to a solid. The crude product was purified by silica gel chromatography (1:19, ethyl acetate:hexanes) to afford 1.26 g of the title compound (81% yield). ¹³C NMR (100 MHz, CDCl₃) 27.8, 43.3, 83.6, 120.3, 127.1, 131.8, 145.7.

Preparation 23

General Procedure for the Copper-Mediated Coupling to Afford Compounds 1 of the Invention

### EXAMPLE 26

### 1-[4-(2-Methyl-oxazol-4-yl)phenyl]-3-[2-(4-methyl-piperazin-1-yl)-pyridin-3-ylmethyl]-piperidin-2-one

A mixture of 3-[2-(4-methyl-piperazin-1-yl)-pyridin-3-ylmethyl]-piperidin-2-one (170 mg, 0.59 mmol), 4-(4-bromo-phenyl)-2-methyl-oxazole (281 mg, 1.2 mmol), copper (I) iodide (45 mg, 0.24 mmol), potassium carbonate (166 mg, 1.2 mmol), and N,N′-dimethylthylendiamine (51 μl, 0.48 mmol) in toluene (1.5 mL) was stirred at 100° C. for 24 h. Copper (I) iodide (45 mg, 0.24 mmol) and N,N′-dimethylethylendiamine (51 μl, 0.48 mmol) were added and the reaction mixture was heated at 100° C. for an additional 24 h. The mixture was cooled to room temperature, poured into water and extracted with dichloromethane. The combined organic extracts were dried (sodium sulfate) and concentrated to provide 450 mg crude product. Purification by silica gel chromatography (12:88, methanol:dichloro-ethane) afforded 107 mg (41% yield) of the title compound. MS (AP/CI) 446.3 (M+H). ¹³C NMR (100 MHz, CDCl₃) 14.2, 22.3, 26.2, 33.7, 42.3, 46.3, 50.5, 51.7, 55.6, 118.8, 126.3, 126.5, 127.8, 129.7, 133.5, 139.1, 140.3, 143.2, 146.1, 162.1, 162.3, 172.5. The enantiomers were separable by HPLC: 65/35 Heptane/Ethanol; Chiralpak AD, 5 cm×50 cm; 85 mL/min). Approximate retention times: t₁=23 min; t₂=33 min.

The following compounds were made using the same general procedure as for Example 26.

### EXAMPLE 27

1-[4-(2-tert-Butyl-oxazol-4-yl)-phenyl]-3-[2-(4-methyl-piperazin-1-yl)pyridin-3-ylmethyl]-piperidin-2-one: MS (AP/CI) 488.3 (M+H). ¹³C NMR (100 MHz, CDCl₃) 22.3, 26.2, 28.8, 33.6, 34.0, 42.3, 46.4, 50.6, 51.7, 55.7, 118.8, 126.5, 127.8, 130.1, 133.1, 139.1, 139.9, 143.1, 146.1, 162.4, 171.8, 172.5. The enantiomers were separable by HPLC: 60/40 Heptane/Ethanol; Chiralpak AD, 5 cm×50 cm; 75 mL/min). Approximate retention times: t₁=12 min; t₂=20 min.

### EXAMPLE 28

1-[4-(2-Isopropyl-oxazol-4-yl)-phenyl]-3-[2-(4-methyl-piperazin-1-yl)pyridin-3-ylmethyl]-piperidin-2-one: MS (AP/CI) 474.2 (M+H). ¹³C NMR (100 MHz, CDCl₃) 20.7, 22.3, 26.1, 28.8, 33.6, 42.3, 46.4, 50.6, 51.7, 55.7, 118.8, 126.4, 126.5, 127.8, 129.9, 133.2, 139.1, 140.0, 143.1, 146.1, 162.3, 169.5, 172.5.

### EXAMPLE 29

1-[4-(2,5-Dimethyl-oxazol-4-yl)-phenyl]-3-[2-(4-methyl-piperazin-1-yl)pyridin-3-ylmeth-yl]-piperidin-2-one: MS (AP/CI) 460.3 (M+H). ¹³C NMR (100 MHz, CDCl₃) 12.0, 14.1, 22.3, 26.2, 33.7, 42.2, 46.4, 50.6, 51.7, 55.7, 118.8, 126.4, 127.4, 127.8, 131.0, 134.0, 139.1, 142.4, 143.8, 146.1, 159.3, 162.3, 172.5. The enantiomers were separable by HPLC: 60/40 Heptane/Ethanol; Chiralpak AD, 5 cm×50 cm; 75 mL/min). Approximate retention times: t₁=13 min; t₂=26 min.

### EXAMPLE 30

3-[2-(4-Methyl-piperazin-1-yl)-pyridin-3-ylmethyl]-1-[4-(tetrahydro-pyran-4-yl)-phenyl]-piperidin-2-one: MS (AP/CI) 449.5 (M+H). ¹³NMR (100 MHz, CDCl₃) 22.3, 26.2, 33.7, 34.1, 41.4, 42.2, 46.2, 50.4, 51.9, 55.5, 68.6, 118.8, 126.4, 127.7, 127.8, 139.2, 141.8, 144.5, 146.1, 162.3, 172.5. The enantiomers were separable by HPLC: Methanol; Chiralpak AD, 10 cm×50 cm; 250 mL/min). Approximate retention times: t₁=25 min; t₂=44 min.

### EXAMPLE 31

3-[2-(4-Methyl-piperazin-1-yl)-pyridin-3-ylmethyl]-1-[4-(tetrahydropyran-4-yl)-phenyl]-pyrrolidin-2-one: MS (AP/CI) 435.5 (M+H). ¹³C NMR (100 MHz, CDCl₃) 24.8, 32.9, 34.1, 41.2, 43.8, 46.4, 46.9, 50.6, 55.6, 68.5, 118.9, 120.1, 127.2, 127.3, 137.9, 138.7, 142.4, 146.3, 162.3, 175.4.

### EXAMPLE 32

3-[2-(4-Methyl-piperazin-1-yl)-pyridin-3-ylmethyl]-1-(4-oxazol-2-yl-phenyl)-pyrrolidin-2-one: MS (AP/CI) 418.4 (M+H). ¹³C NMR (100 MHz, CDCl₃) 24.7, 33.0, 43.9, 46.4, 46.7, 50.7, 55.6, 118.9, 119.5, 123.5, 127.1, 127.2, 128.6, 138.7, 141.4, 146.4, 161.8, 162.2, 175.8.

### EXAMPLE 33

3-[2-(4-Methyl-piperazin-1-yl)pyridin-3-ylmethyl]-1-(4-oxazol-4-yl-phenyl)-pyrrolidin-2-one: MS (AP/CI) 418.4 (M+H). ¹³C NMR (100 MHz, CDCl₃) 24.8, 33.0, 43.9, 46.4, 46.8, 50.7, 55.6, 118.9, 119.9, 126.2, 127.0, 127.2, 133.7, 138.7, 139.5, 140.1, 146.4, 151.5, 162.3, 175.6.

### EXAMPLE 34

3-[2-(4-Methyl-piperazin-1-yl)-pyridin-3-ylmethyl]-1-(4-oxazol-5-yl-phenyl)-pyrrolidin-2-one: MS (AP/CI) 418.4 (M+H). ¹³C NMR (100 MHz, CDCl₃) 24.7, 33.0, 43.9, 46.3, 46.7, 50.6, 55.5, 118.9, 119.9, 121.3, 123.9, 125.1, 127.1, 138.7, 139.9, 146.4, 150.5, 151.3, 162.2, 175.7.

### EXAMPLE 35

1-[4-(2-Methyl-oxazol-4-yl)phenyl]-3-[2-(4-methyl-piperazin-1-yl)-pyridin-3-ylmethyl]-pyrrolidin-2-one: ¹³C NMR (100 MHz, CDCl₃) 14.2, 24.8, 33.1, 43.9, 46.3, 46.8, 50.6, 55.6, 118.9, 119.9, 126.0, 127.2, 127.5, 133.2, 138.7, 139.3, 140.3, 146.4, 162.1, 162.2, 175.5. MS (AP/CI) 432.4 (M+H).

### EXAMPLE 36

1-[4-(1-Methoxy-cyclobutyl)-phenyl]-3-[2-(4-methyl-piperazin-1-yl)-pyridin-3-ylmethyl]-piperidin-2-one: MS (AP/CI) 449.3 (M+H). ¹³C NMR (100 MHz, CDCl₃) 13.1, 22.3, 26.2, 33.0, 33.1, 33.8, 42.1, 46.4, 50.6, 50.8, 51.8, 55.8, 81.4, 118.7, 126.1, 127.3, 127.7, 139.1, 141.7, 142.6, 146.1, 162.3, 172.6.

Preparation 24

General Procedure for Palladium Mediated Coupling to Afford Compounds 1 of the Invention

### EXAMPLE 37

3-[2-(4-Methyl-piperazin-1-yl)-pyridin-3-ylmethyl]-1-(4-trifluoromethyl-phenyl)-pyrrolidin-2-one. 3-[2-(4-Methyl-piperazin-1-yl)-pyridin-3-ylmethyl]-pyrrolidin-2-one (600 mg, 2.2 mmol), 4-bromobenzotriflouride (369 μL, 2.6 mmol), tris(dibenzylideneacetone)dipalladium (0) (100 mg, 0.11 mmol), 9,9-dimethyl-4,5-bis(diphenylphosphino)xanthene (191 mg, 0.33 mmol) and cesium carbonate (1.08 g, 3.3 mmol) were combined in 4 ml dioxane and heated at 100° C. for 18 h. The cooled reaction mixture was then poured into water and extracted with dichloromethane. The combined organic extracts were dried (Na₂SO₄) and concentrated to an oil. Purification by silica gel chromatography (8:92, methanol: dichloromethane) afforded 500 mg (54% yield) of the title compound as an oil. MS (AP/CI) 419.3 (M+H). ¹³C NMR (100 MHz, CDCl₃) 24.7, 33.0, 43.8, 46.3, 46.6, 50.7, 55.6, 118.9, 119.2, 126.1, 126.2, 127.0, 138.7, 142.6, 146.5, 162.2, 176.0.

The following compounds were prepared using the same general procedure as Example 37:

### EXAMPLE 38

1-[4-(1-Hydroxy-cyclopentyl)phenyl]-3-[2-(4-methyl-piperazin-1-yl)-pyridin-3-ylmethyl]-pyrrolidin-2-one: MS (AP/CI) 435.3 (M+H). ¹³C NMR (100 MHz, CDCl₃) 24.0, 24.8, 33.0, 42.0, 43.8, 46.2, 46.9, 50.5, 55.5, 83.4, 118.9, 119.7, 125.9, 127.2, 138.2, 138.7, 143.6, 146.4, 162.2, 175.5.

### EXAMPLE 39

1-[4-(1-Hydroxy-1-methyl-ethyl)-phenyl]-3-[2-(4-methyl-piperazin-1-yl)-pyridin-3-ylmethyl]-pyrrolidin-2-one: MS (AP/CI) 409.3 (M+H). ¹³C NMR (100 MHz, CDCl₃) 24.8, 32.0, 32.9, 43.8, 46.3, 46.9, 50.5, 55.5, 72.2, 118.9, 119.7, 125.2, 127.2, 138.0, 138.7, 145.9, 146.3, 162.2, 175.5. 50/50 Heptane/Ethanol; Chiralpak AD, 5 cm×50 cm; 75 mL/min). Approximate retention times: t₁=25 min; t₂=34 min.

### EXAMPLE 40

1-(4-tert-Butyl-phenyl)-3-[2-(4-methyl-piperazin-1-yl)-pyridin-3-ylmethyl]-pyrrolidin-2-one: MS (AP/CI) 407.4 (M+H). ¹³C NMR (100 MHz, CDCl₃) 24.8, 31.6, 32.9, 34.6, 43.8, 46.4, 46.9, 50.7, 55.6, 118.8, 119.7, 125.9, 127.3, 137.1, 138.6, 146.3, 147.7, 162.4, 175.3. The enantiomers were separated by HPLC: 75/25 Heptane/Isopropanol; Chiralpak AD, 5 cm×50 cm; 75 mL/min). Approximate retention times: t₁=24 min; t₂=32 min.

### EXAMPLE 41

1-[4-(1-Hydroxy-cyclopentyl)-phenyl]-3-[2-(4-methyl-piperazin-1-yl)-pyridin-3-ylmeth-yl]-piperidin-2-one: MS (AP/CI) 449.5 (M+H). ¹³C NMR (100 MHz, CDCl₃) 22.3, 24.0, 26.2, 33.7, 42.1, 42.2, 46.4, 50.6, 51.9, 55.7, 83.5, 118.8, 126.1, 126.2, 127.8, 139.1, 142.3, 145.7, 146.1, 162.3, 172.6.

### EXAMPLE 42

1-(4-tert-Butyl-phenyl)-3-[2-(4-methyl-piperazin-1-yl)-pyridin-3-ylmethyl]-piperidin-2-one: MS (AP/CI) 421.5 (M+H). ¹³C NMR (100 MHz, CDCl₃) 22.3, 26.2, 31.6, 33.7, 34.7, 42.1, 46.4, 50.6, 51.8, 55.7, 118.7, 125.8, 126.3, 127.8, 139.1, 141.0, 146.1, 149.7, 162.3, 172.5.

### EXAMPLE 43

3-[2-(4-Methyl-piperazin-1-yl)-pyridin-3-ylmethyl]-1-phenyl-piperidin-2-one: MS (AP/CI) 365.4 (M+H). ¹³C NMR (100 MHz, CDCl₃) 22.3, 26.2, 33.7, 42.2, 46.3, 50.5, 51.9, 55.6, 118.8, 126.4, 126.9, 127.8, 129.4, 139.1, 143.7, 146.1, 172.5.

### EXAMPLE 44

3-[2-(4-Methyl-piperazin-1-yl)-pyridin-3-ylmethyl]-1-(4-trifluoromethoxy-phenyl)-piperidin-2-one: MS (AP/CI) 449.4 (M+H). ¹³C NMR (100 MHz, CDCl₃) 22.3, 26.2, 33.7, 42.1, 46.4, 50.7, 51.8, 55.7, 118.8, 121.9, 127.6, 127.8, 139.1, 142.1, 146.2, 162.4, 172.7.

### EXAMPLE 45

1-[4-(1-Hydroxy-1-methyl-ethyl)-phenyl]-3-[2-(4-methyl-piperazin-1-yl)pyridin-3-ylmethyl]-piperidin-2-one: MS (AP/CI) 423.5 (M+H). ¹³C NMR (100 MHz, CDCl₃) 22.3, 26.1, 31.9, 33.6, 42.2, 46.3, 50.5, 51.9, 55.6, 72.4, 118.9, 125.6, 126.0, 127.9, 139.2, 142.0, 146.1, 148.0, 162.3, 172.6. The enantiomers were separated by HPLC: 70/30 Heptane/Isopropanol/0.1% Trifluoroacetic acid; Chiralpak AD, 5 cm×50 cm; 75 mL/min). Approximate retention times: t₁=19 min; t₂=31 min. Additional silica gel chromatography required to remove olefin: 91.5: 8: 0.5, dichloromethane: methanol: ammonium hydroxide.

### EXAMPLE 46

1-[4-(1-Hydroxy-cyclobutyl)-phenyl]-3-[2-(4-methyl-piperazin-1-yl)-pyridin-3-ylmethyl]-piperidin-2-one: MS (AP/CI) 435.5 (M+H). ¹H NMR (400 MHz, CDCl₃) □1.42-1.57 (m, 1H), 1.63-1.91 (m, 3H), 1.92-2.03 (m, 2H), 2.15 (br, 1H), 2.36 (s, 3H), 2.30-2.39 (m, 2H), 2.51-2.68 (m, 5H), 2.75 (dd, 1H, J=14.1 and 10.2 Hz), 2.93-3.01 (m, 1H), 3.08-3.25 (m, 4H), 3.50 (dd, 1H, J=14.2 and 3.7 Hz), 3.60-3.70 (m, 2H), 6.91 (dd, 1H, J=7.1 and 4.6 Hz), 7.26 (d, 1H, J=8.2 Hz), 7.49 (dd, 1H, J=7.5 and 1.7 Hz), 7.53 (d, 1H, J=8.3 Hz), and 8.20 (dd, 1H, J=4.9 and 1.6 Hz).

What is claimed is:

1. A compound having the structural formula: 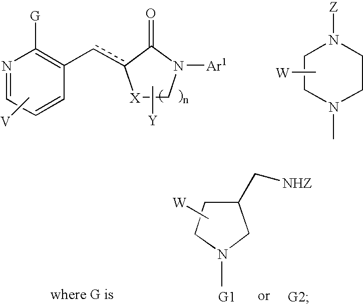
where the dashed line represents an optional double bond;
Ar¹ is phenyl, a 5- or 6-membered heteroaryl ring, or an 8- to 10-membered fused aryl or heteroaryl ring system, said heteroaryl ring, and the heteroaryl moiety of said heteroaryl ring system comprising an aromatic ring made up of carbon and from one to four atoms of other elements selected independently from the group consisting of oxygen, nitrogen, and sulfur, which Ar¹ may be singly or multiply substituted with, independently, halogen, hydroxy, nitro, cyano, R¹, R², R³, —OR₄, —OC(═O)R⁵, —COOR⁶, NHR⁷, NR⁸R⁹, —NHC(═O)R¹⁰, N(R¹¹)(C═O)R¹², —C(═O)NHR¹³, or Ar²;
X is CH₂, NH, or O; V, W, and Y are, independently, hydrogen, halogen, hydroxy, nitro, cyano or R⁷,
R¹-R¹³ are, independently, C₁-C₈alkyl, C₂-C₈ alkenyl, C₁-C₈alkoxy, C₁-C₈ hydroxyalkyl, C₁-C₈ alkenoxy, said alkyl, alkenyl, alkoxy, or alkenoxy optionally substituted with one or more halogen atoms or nitro, cyano, or hydroxyl groups, said alkyl or alkenyl groups being straight-chain, branched, or cyclic, wherein an alkoxy-substituted alkyl group may form a cyclic ether, or, in the case of NR⁸R⁹, R⁸ and R⁹ may be linked together to form an additional ring;
Z is C₁-C₆ alkyl or C₁-C₆ alkylcarbonyl;
Ar² is a 5- or 6-membered aryl or heteroaryl ring or an 8 to 10-membered fused aryl or heteroaryl ring system, which Ar² may be singly or multiply substituted with, independently, halogen, hydroxy, nitro, cyano, R¹, R², R³, OR⁴, OC(═O)R⁵, COOR⁶, NHR⁷, NR⁸R⁹, NHC(═O)R¹⁰, N(R¹¹)(C═O)R¹², C(═O)NHR¹³; and
n is 1 or 2;
or a pharmaceutically acceptable salt thereof.

2. The compound according to claim 1 wherein V and Y are hydrogen, X is CH₂, and G is 4-methyl-piperazin-1-yl.

3. The compound according to claim 2, wherein Ar¹ is phenyl.

4. The compound according to claim 3, wherein Ar¹ is substituted with a phenyl or a monocyclic heteroaryl group.

5. The compound according to claim 3, wherein Ar¹ is substituted with R¹, wherein R¹ is a cycloalkyl or saturated cyclic ether group.

6. The compound according to claim 1, wherein said compound is selected from the group consisting of:
1-[4-(3,5-Dimethyl-isoxazol-4-yl)-phenyl]-3-[2-(4-methyl-piperazin-1-yl)-pyridin-3-ylmethylene]-piperidin-2-one;
2-[2-(4-Methyl-piperazin-1-yl)-pyridin-3-ylmethylene]-4-[4-(tetrahydro-pyran-4-yl)-phenyl]-morpholin-3-one;
1-[4-(3,5-Dimethyl-isoxazol-4-yl)phenyl]-3-[2-(4-methyl-piperazin-1-yl)-pyridin-3-ylmethyl]-piperidin-2-one;
2-[2-(4-Methyl-piperazin-1-yl)-pyridin-3-ylmethyl]-4-[4-(tetrahydro-pyran-4-yl)-phenyl]-morpholin-3-one;
1-[4-(2-Methyl-oxazol-4-yl)phenyl]-3-[2-(4-methyl-piperazin-1-yl)-pyridin-3-ylmethyl]-piperidin-2-one;
1-[4-(2-tert-Butyl-oxazol-4-yl)phenyl]-3-[2-(4-methyl-piperazin-1-yl)-pyridin-3-ylmethyl]-piperidin-2-one;
1-[4-(2-Isopropyl-oxazol-4-yl)-phenyl]-3-[2-(4-methyl-piperazin-1-yl)pyridin-3-ylmethyl]-piperidin-2-one;
1-[4-(2,5-Dimethyl-oxazol-4-yl)-phenyl]-3-[2-(4-methyl-piperazin-1-yl pyridin-3-ylmethyl]-piperidin-2-one;
3-[2-(4-Methyl-piperazin-1-yl)-pyridin-3-ylmethyl]-1-[4-(tetrahydro-pyran-4-yl)-phenyl]-piperidin-2-one;
3-[2-(4-Methyl-piperazin-1-yl)-pyridin-3-ylmethyl]-1-[4-(tetrahydro-pyran-4-yl)-phenyl]-pyrrolidin-2-one;
3-[2-(4-Methyl-piperazin-1-yl)-pyridin-3-ylmethyl]-1-(4-oxazol-2-yl-phenyl)-pyrrolidin-2-one;
[2-(4-Methyl-piperazin-1-yl)-pyridin-3-ylmethyl]-1-(4-oxazol-4-yl-phenyl)-pyrrolidin-2-one;
3-[2-(4-Methyl-piperazin-1-yl)-pyridin-3-ylmethyl]-1-(4-oxazol-5-yl-phenyl)-pyrrolidin-2-one;
1-[4-(2-Methyl-oxazol-4-yl)-phenyl]-3-[2-(4-methyl-piperazin-1-yl)-pyridin-3-ylmethyl]-pyrrolidin-2-one;
1-[4-(1-Methoxy-cyclobutyl)-phenyl]-3-[2-(4-methyl-piperazin-1-yl)-pyridin-3-ylmethyl]-piperidin-2-one;
3-[2-(4-Methyl-piperazin-1-yl)-pyridin-3-ylmethyl]-1-(4-trifluoromethyl-phenyl)-pyrrolidin-2-one;
1-[4-(1-Hydroxy-cyclopentyl)phenyl]-3-[2-(4-methyl-piperazin-1-yl)-pyridin-3-ylmethyl]-pyrrolidin-2-one;
1-[4-(1-Hydroxy-1-methyl-ethyl)phenyl]-3-[2-(4-methyl-piperazin-1-yl)pyridin-3-ylmethyl]-pyrrolidin-2-one;
1-(4-tert-Butyl-phenyl)-3-[2-(4-methyl-piperazin-1-yl)-pyridin-3-ylmethyl]-pyrrolidin-2-one;
1-[4-(1-Hydroxy-cyclopentyl)phenyl]-3-[2-(4-methyl-piperazin-1-yl)-pyridin-3-ylmethyl]-piperidin-2-one;
1-(4-tert-Butyl-phenyl)-3-[2-(4-methyl-piperazin-1-yl)-pyridin-3-ylmethyl]-piperidin-2-one;
3-[2-(4-Methyl-piperazin-1-yl)-pyridin-3-ylmethyl]-1-phenyl-piperidin-2-one;
3-[2-(4-Methyl-piperazin-1-yl)-pyridin-3-ylmethyl]-1-(4-trifluoromethoxy-phenyl)-piperidin-2-one;
1-[4-(1-Hydroxy-1-methyl-ethyl)phenyl]-3-[2-(4-methyl-piperazin-1-yl)-pyridin-3-ylmethyl]-piperidin-2-one; and
1-[4-(1-Hydroxy-cyclobutyl)-phenyl]-3-[2-(4-methyl-piperazin-1-yl)-pyridin-3-ylmethyl]-piperidin-2-one.

7. A pharmaceutical composition comprising a pharmaceutically effective amount of the compound of claim 1, or of a pharmaceutically acceptable salt thereof, and a pharmaceutically effective carrier.

8. A method of treating a disorder selected from the group consisting of anxiety, depression, dysthymia, major depressive disorder, migraine, post-traumatic stress disorder, avoidant personality disorder, borderline personality disorder and phobias in a patient, comprising administering to a patient in need of treatment an effective amount of a compound of claim 1, or a pharmaceutically acceptable salt thereof, and a pharmaceutically effective carrier.

9. A method of treating migraine, headache or cluster headache in a patient in need of treatment, comprising administering to said patient an amount of a compound of claim 1, or a pharmaceutically acceptable salt thereof, that is effective in treating such disorder.

10. A method of treating a disorder selected from the group consisting of anxiety, depression, dysthymia, major depressive disorder, migraine, post-traumatic stress disorder, avoidant personality disorder, borderline personality disorder and phobias in a patient, comprising administering to a patient in need of treatment a compound of claim 1, or a pharmaceutically acceptable salt thereof, and a serotonin reuptake inhibitor, wherein the amount of the active compounds are such that the combination is effective in treating the disorder.

11. A method of treating migraine, headache, or cluster headache in a patient in need of treatment, comprising administering to said patient a compound of claim 1, or a pharmaceutically acceptable salt thereof, and a serotonin reuptake inhibitor, wherein the amount of the active compounds are such that the combination is effective in treating the disorder.

12. A method of visualizing a 5-HT_{1B}-containing organ in a mammal, comprising administering to said mammal a radioactive form of a compound according to claim 1, and detecting the emissions of the radioactive compound.

13. An intermediate for the synthesis of a compound of Formula I, selected from the group consisting of:
3-[2-(4-Methyl-piperazin-1-yl)-pyridin-3-ylmethylene]-pyrrolidin-2-one;
3-[2-(4-Methyl-piperazin-1-yl)-pyridin-3-ylmethylene]-piperidin-2-one; and,
3-[2-(4-Methyl-piperazin-1-yl)-pyridin-3-ylmethylene]-morphilin-2-one.
